(19) United States
(12) Patent Application Publication
(10) Pub. No.: US 2009/0146081 A1
(43) Pub. Date: Jun. 11, 2009

(54) Surface Plasmon Enhanced Radiation Methods and Apparatus

(75) Inventor: Peter Randolph Hazard Stark, Andover, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 12/087,301

(22) PCT Filed: Jan. 5, 2007
(86) PCT No.: PCT/US2007/000458

§ 371 Date: Oct. 10, 2008

Related U.S. Application Data
(60) Provisional application No. 60/757,392, filed on Jan. 9, 2006.
(60) Provisional application No. 60/757,215, filed on Jan. 6, 2006.

(51) Int. Cl. A61N 5/00; G21G 4/00

(52) U.S. Cl. 250/492.2; 250/493.1

(57) ABSTRACT

Methods and apparatus in which a plurality of independently controllable surface emitting lasers (SELs) are controlled to generate radiation that irradiates a plurality of surface plasmon enhanced illumination (SPEI) apparatus. The irradiated SPEI apparatus in turn generate surface plasmon enhanced radiation that may be employed for a variety of applications, including maskless (i.e., “direct write”) photolithography techniques in which a photoresist is exposed to individually controllable beams of surface plasmon enhanced radiation.

### FIELD OF THE DISCLOSURE

This disclosure relates to methods and apparatus in which target areas are illuminated with one or more spots or lines of light having very small dimensions and the use of these spots or lines of light and changes to them as a sensing technique.

### BACKGROUND

Typical optical microscopy, far-field light microscopy, cannot resolve distances less than the Rayleigh limit. The Rayleigh criterion states that two images are regarded as just resolved when the principal maximum (of the Fraunhofer diffraction pattern) of one coincides with the first minimum of the other [see Born, M. and Wolf, E. Principles of Optics. Cambridge University Press 6^{th} ed. p. 415 (1980)]. For a circular aperture, this occurs at

$$w = 0.61 \frac{λ}{NA} .$$

For example, the wavelength (λ) at the peak emission of a green fluorescent protein (EGFP) is 508 nm. Hence, for a very high numerical aperture (NA) of the objective, NA of 1.4, the minimum separation (w) that can be resolved in a GFP labeled sample is 221 nm. Currently, there are several possible methods for achieving resolution of spatial locations of proteins below the Rayleigh limit. They include: Confocal Microscopy, Fluorescence Resonance Energy Transfer (FRET), Atomic Force Microscopy (AFM), Near-Field Scanning Optical Microscopy (NSOM), Harmonic Excitation Light-Microscopy (HELM), Stimulated Emission Depletion Microscopy (STED-Microscopy) and Electron Microscope Immunocytochemistry.

Confocal Microscopy is a technique in which a very small aperture(s) is/are placed in the optical path to eliminate any unfocused light. This allows for a substantial increase in signal to noise ratio over conventional light microscopy. Also, it is possible to reduce the width of the central maximum of the Fraunhoffer pattern using a small slit or aperture. This, in turn allows a substantially enhanced resolution of 1.4 times better than the Rayleigh limit. Therefore, with this method, using the above protein as an example, a spatial resolution of 156 nm is achieved.

Typical confocal microscopy is not without disadvantages. By increasing the signal to noise ratio by decreasing the aperture size, the total signal level decreases concurrently. To bring the signal back to a useful level, the input power level must be increased. This, in turn, not only can cause photo-bleaching in the fluorophores at which one intends to look but also the surrounding area where the light is incident, just not collected. A method around this is to use two-photon excitation. Fluorescence from the two-photon effect depends on the square of the incident light intensity, which in turn, decreases approximately as the square of the distance from the focus. Because of this highly nonlinear (˜fourth power) behavior, only those dye molecules very near the focus of the beam are excited, while the surrounding material is bombarded only by comparatively much fewer of the low energy photons, which are not of enough energy to cause photo bleaching. Multi-photon excitation requires highly skilled technicians and is somewhat expensive for clinical use. Because it acquires only a small area at once, the surface must be scanned in three dimensions for mapping.

Fluorescence Resonance Energy Transfer (FRET) can provide exquisite resolution of single chromophores. The resonance occurs when one fluorophore in an excited state transfers a portion of its energy to a neighboring chromophore. For this to take place, there must exist some overlap between the emission spectrum of the fluorophore to absorption spectrum of the chromophore (the frequency of the emission spectrum should be somewhat higher than the absorption spectrum of the chromophore). The process does not occur through photonic emission and absorption but through a dipole-dipole interaction. The strength of the interaction varies as r⁻⁶. The Forster distance [see Forster, T Discuss. Faraday Soc. 27 7-29 (1959)] is the distance at which the efficiency of the transfer is such that there exists equal probability that the fluorophore loses energy to radiative decay or dipole-dipole interaction. The Forster distance, essentially, is the threshold at which FRET will no longer exist for a given pair. Typically the Forster distance is between 3 and 6 nm [see Pollok & Heim “Using GFP in FRET-based Applications” Trends in Cell Biology 9 pp 57-60 (1999)].

By placing either of the complementary pair near the other, resolutions of less than the Forster distance can be attained. The problem with this technique in determining relative locations is that one of the pair needs to be located within the resolution tolerances desired for spatial mapping. This can be achieved by placing one of the pair on a probe used in either atomic force microscopy (AFM) or near-field scanning optical microscopy (NSOM). Another problem is that dipole-dipole interactions are dependent-on the relative orientation of the two. To maximize signal from the interaction would require a 3D scan around one of the pair.

Atomic Force Microscopy (AFM) can be envisioned as a very small (usually metal) stylus dragged across a surface giving feedback as to the height, Z, of the stylus relative to the surface. Resolution can be as fine as the scanning step size (typically 5 nm). By scanning across the surface, X and Y coordinates are obtained provided that the origin remains fixed (i.e., that there is no drift in the translation stage due to thermal or other effects). There are many methods for ensuring that the stylus does not actually contact the sample but maintains very accurate resolution of the Z coordinate. Because only surface morphology is measured, differentiating several molecules can be extremely difficult unless the dimension's and orientations of those molecules are well known. A solution to this might be to add tags of discrete lengths or shapes, which could be bound indirectly to the molecules of interest. This method, however, would require that the tissue sample to be planar before the tags were bound to the surface.

To increase the information of AFM, one could use Near-Field Scanning Optical Microscopy (NSOM or SNOM). NSOM uses a principle similar to AFM in which a stylus is scanned over a surface providing topographical information. However, the stylus is a conductor of photons. By emitting light from the tip of the stylus, optical measurements such as fluorescence can be obtained. Most often, these styli are fiber probes that have tapered tips and then are plated with a conductive material (aluminum is most often chosen as its skin depth for optical radiation is quite low, ˜13 nm at 500 nm) with a small aperture where the coating is broken. [See Betzig & Trautman “Near-Field Optics: Microscopy, Spectroscopy, and Surface Modification beyond the Diffraction Limit” Science 257 pp 189-195 (1992)]. Another approach is to use what are called “apertureless probes” [see Sanchez, Novotny and Xie “Near-Field Fluorescence Microscopy Based on Two-Photon Excitation with Metal Tips” Physical Review Letters Vol 82 20 pp 4014-4017 (1999)] where an evanescent wave is excited by bombardment with photons at the tip of a sharpened metal probe. Because the tip can be made very sharp (radii of 5 nm are achievable), resolutions can be correspondingly smaller. An associated problem with the “apertureless probes” is that the probe generates a white light continuum, which significantly decreases the signal to noise ratio.

By making the diameter (assuming a circular geometry) of the emission portion of the tip of the stylus very small (smaller than resolution desired) and keeping the tip to sample distance less than that distance, so that the diffraction is small, a nanometric light source is available. This light source can be used to excite fluorescence in the sample. Because the size of the source is very small and the scanning increments are also very small, highly resolved information on spatial locations of the fluorophores can be gleaned by inspection in the far field. Alternatively, the probe can be used for collection, measuring fluorescence or reflection or even transmission from illumination from the other side of the sample.

Because the aperture size in a conventional probe is so much smaller than the wavelength of the excitation light and only an evanescent mode is supported, very little light is transmitted through the aperture. Diffraction effects limit the effective collimated length from the aperture to less than diameter of the aperture. This, then, requires that the aperture be held below a maximum height above the surface of the sample. Ideally, a fixed height above the surface (usually less than 10 nm) is used for relative contrast measurements. The height of the aperture relative to the surface is kept contrast by measuring the shear force on the tip of the probe or by optical methods and is modulated to maintain that height. For this reason, NSOM is particularly susceptible to vibrations and experimental work requires isolation platforms.

Scanning the surface takes a fair amount of time. Thermal drift in commercially available open and closed loop nanometric scanning stages is about 20-30 nm/min. [see Frohn, Knapp and Stemmer “True optical resolution beyond the Rayleigh limit achieved by standing wave illumination” Proceedings of the National Academies of Science Vol. 97, 13 pp 7232-7236 (2000)]. This can be severely limiting if scanning time is more than a few tens of seconds and resolution less than 50 nm is desired. If the surface is scanned for several different types of molecules, the required time to investigate a single cell becomes far too large for use in a clinical setting and would require multiple homings of the scanning stage. An approach to diminishing the scanning time may be to scan with multiple probes concurrently. This approach would be limited to just a few probes as on a small (20² μm²) surface, the relatively large size of the probes' bodies would interfere mechanically with each other.

U.S. Pat. Nos. 5,973,316 and 6,052,238 issued to Ebbesen et al. of the NEC Research Institute, Inc. describe a NSOM device which employs an array of subwavelength apertures in a metallic film or thin metallic plate. Enhanced transmission through the apertures of the array is greater than the unit transmission of a single aperture and is believed to be due to the active participation of the metal film in which the aperture array is formed. In addition to enhancing transmission, the array of apertures reduces scanning time by increasing the number of nanometric light sources.

A second method for increasing the number of light sources illuminates the sample with a mesh-like interference pattern and by post processing of the images. In Harmonic Excitation Light Microscopy (HELM), a laser is split into four beams and two of those beams modulated to produce an extended two-dimensional interference field with closely spaced antinodes. By introducing the beams at an angle to the surface to be imaged, an effective offset in reciprocal space is produced around an origin. If four images are taken around this origin and one at the origin, it is possible to construct, with post processing, a smaller single antinode which acts as a nanometric light source. This process can result in a lateral resolving power of close to 100 nm or half of the Rayleigh distance for green light. Because only a few images are required to map an entire surface, the acquisition time is extremely short (around 1.6 s for a 25 μm×25 μm area with 100 nm resolution.) Due to the required precision in the location of the four images around the origin and the drift associated with the scanning stage, it is unlikely that the resolution will be dramatically increased.

Another new form of microscopy is that introduced by Klar et al. [see Klar, Jakobs, Dyba, Egner and Hell “Fluorescence microscopy with diffraction resolution barrier” Proceedings of the National Academies of Science Vol 97 15 pp 8206-8210 (2000)] called Stimulated Emission Depletion (STED) Microscopy. STED microscopy is based on a method of quenching fluorescence by stimulated emission depletion reducing the fluorescing spot size. [See Hell & Wichmann “Breaking the Diffraction Resolution Limit by Stimulated-Emission-Depletion Fluorescence Microscopy” Opt. Lett 19 11 780-782 (1994); Lakowicz, Gryczynski, Bogdanov and Kusba. “Light Quenching and Fluorescence Depolarization of Rhodamine-B and Applications of this Phenomenon to Biophysics” J. Phys. Chem. 98 1 334-342 (1994); Hell, S. W. Topics in Fluorescence Spectroscopy, ed. Lakowicz (Plenum Press, NY), Vol. 5, pp. 361-422; and Klar & Hell “Subdiffraction resolution in far-field fluorescence microscopy” Opt. Lett 24 14, 954-956 (1999)]. Fluorescence can be quenched by subjecting a fluorophore to light at the lower energy edge (red side) of its emission spectrum. This forces the fluorophore to a higher vibrational level of the ground state, which, by decay of that state prevents re-excitation. Fluorescence can be turned on, with an ordinary excitation source, and turned off, with the STED beam, at will. By introducing an interference pattern in the STED beam, a local set of maxima and minima can be created. If the maxima of the STED beam are overlaid onto the fluorescence induced by the excitation beam, the fluorescence is quenched. However, where the minima occur, fluorescence continues. The fluorescing spot size is controlled by the union of the minimum or minima of the STED beam and the maximum of the excitation beam. Because STED is nonlinear with intensity, the sharpness of the minimum, maximum transition can be effectively increased allowing a narrow, almost delta behavior to be displayed. This, however, can result in severe photo stress to the sample and, possibly, dual photon effects, causing competing modes in the area where quenching is desired. So far, resolution in the radial (X, Y) direction is around 100 nm, but there is no reason to expect that the resolution can't be substantially improved. Once again, though, STED microscopy is a scanning type and will suffer from the same drawbacks all scanning instruments do, (e.g., thermal drift, vibration problems, registration of near field excitement with far field collection and scan time.)

### SUMMARY

The present disclosure contemplates a different technique to achieve sub-Rayleigh criterion resolution, which is here called “Surface Plasmon Enhanced Illumination” (SPEI). SPEI is related to NSOM in that nanometric light sources are created by subwavelength apertures. By applying the principles of the present disclosure, a significant reduction in the size of the area illuminated by each aperture is achieved, resulting in significantly improved resolution.

The present disclosure takes the form of methods and apparatus that employ novel physical structures to provide nanometric spot or line illumination. In accordance with the disclosure, one or more apertures are formed through a first planar conductive material. Each aperture (which may be either a hole or a slit) has at least one cross-sectional dimension which is less than the wavelength of light which is incident to the planar material. In accordance with a feature of the disclosure, the structure includes means for confining the electronic excitation induced in that portion of the planar surface near the end of the aperture from which the light exits.

The conductive plane that receives the incident light may be placed on one outer surface of a dielectric material. The dielectric material's interface with the conductive plane that receives the incident light establishes a substantially different effective dielectric function in that interface than that of the conductive plane that receives the incident light. This difference in effective dielectric function prevents the excitation of large densities of surface plasmons in non-illuminated plane of the metal if monochromatic light is used at the resonant wavelength of the illuminated metallic plane. Therefore light should not be substantially emitted from the non-illuminated metallic plane.

Alternatively, the sidewalls of the aperture may be conductive to conduct excitation currents and act as a pseudo-waveguide for the light traveling through the aperture. At the exit end of the aperture, the amount of exposed conductive material is limited to an area immediately surrounding the hole exit by a dielectric-material, or by a groove cut into the surface of the conductive material at the exit plane to a depth substantially deeper than the skin depth of the induced excitation and of such width and spacing to prevent an unwanted resonance of surface plasmons in that surface.

The present disclosure substantially reduces, compared to an array of subwavelength apertures in a monometallic film such as those described by Ebbesen et al., the size of the area of illumination produced by each aperture using the combination of a metallic layer on which surface plasmons are induced by incident light and surface composed of a material of substantially different dielectric function, such as an insulator or a different metal, so that the excitation of the surface plasmons in the light emitting surface in the exit surface layer will be different than those excited in the metallic layer that is excited by the incident light, and only the light from the decaying resonant surface plasmons of the exit layer will emit from that surface. The photons associated with the resonance of the incident or upper surface will be constrained to exit from the hole itself or from the walls of the hole.

In accordance with the disclosure, the light barrier comprises an illuminated surface consisting of a continuous conductive metallic layer in combination with an exit layer having substantially different dielectric properties. One or more apertures through the barrier (one or more holes or slits) then form “photonic funnels” through the barrier. Note that confining or eliminating electronic surface excitation on the surface opposite to the illuminated surface works with a single aperture as well as an array of apertures.

The disclosure may advantageously take the form of an array of apertures (holes or slits) formed in structure consisting of a dielectric substrate coated with a conductive metal film on one or both surfaces, or by a thick metallic film, and which further incorporates means for confining the electronic surface excitation to an area immediately adjacent to the apertures where light exits the structure. The means for confining the electronic surface excitation preferably takes the form of a layer of material having dielectric properties that differ substantially from those of the illuminated metal layer, and may consist of a dielectric insulator, a “bad metal” having different dielectric properties, grooves or surface irregularities at the exit-surface, or a combination of these. The structure which confines the electronic surface excitation restricts the size of the spot or line of illumination from each aperture, and the use of an array of apertures, or an array of surface irregularities on the metal film, increases the intensity of the illumination from each aperture

The present disclosure may also be applied to advantage in a direct write photolithography system in which the small illumination spot size resulting from the non-evanescent, collimated illumination that propagates from each aperture alters the physical properties of a photoresist that is moved relative to the apertures by a transport mechanism. All of the apertures in an array may produce illumination that varies in the same way to produce the same illuminated pattern on the photoresist in the vicinity of each aperture, or a separate controllable light source may be used to illuminate the conductive surface of the light barrier in the vicinity of each aperture to thereby control the intensity of the small spot illumination from each aperture. The light sources may be implemented as an array of controllable semiconductor light sources, such as light emitting diodes or surface emitting lasers.

The present disclosure may also be applied to advantage in an optical data storage device. Several arrangements may be devised for combining the hole array with some medium for data storage. A light source, such as a laser, may be directed onto the front surface of the hole array which collects and funnels the array of light onto an optical storage medium. The bit value stored at each position in the storage medium controls the propagation of light through the storage medium to an adjacent pixel position in a charge coupled device (CCD) or other area detectors. A translation mechanism effects movement of the storage medium relative to the hole array in incremental steps, with each step distance being equal to the aperture size. In an alternative arrangement, data may be represented by illumination levels, such as gray scale values or color levels, and optical means may be used in place of or to supplement the mechanical translation mechanism.

The well defined and highly concentrated areas of illumination created by using such a structure as a light source provide significant advantages in microscopy and in optical data storage devices. The confined illumination patterns produced in accordance with the disclosure may be used to construct a “Surface Plasmon Enhanced Microscope” (SPEM) exhibiting markedly improved resolution, to construct an optical data storage device capable of storing larger amounts of data in optical storage media with much higher data access rates than is achievable with current optical data storage devices, and to provide a high throughput photolithography technique that can be applied to advantage in semiconductor fabrication and patterning for self-assembly and biological applications.

In sum, one embodiment of the present disclosure is directed to an apparatus, comprising a plurality of surface emitting lasers (SELs), and a plurality of surface plasmon enhanced illumination (SPEI) apparatus. The SPEI apparatus are disposed with respect to the plurality of SELs such that a first SEL of the plurality of SELs, configured to generate first radiation, irradiates at least a first-SPEI apparatus of the plurality of SPEI apparatus when the first radiation is generated, and a second SEL of the plurality of SELs, configured to generate second radiation, irradiates at least a second SPEI apparatus of the plurality of SPEI apparatus when the second radiation is generated.

Another embodiment is directed to a photolithography method, comprising: generating first radiation from a first surface emitting laser (SEL); irradiating at least a first surface plasmon enhanced illumination (SPEI) apparatus with the first radiation so as to generate first surface plasmon enhanced radiation; generating second radiation from a second surface emitting laser (SEL); irradiating at least a second surface plasmon enhanced illumination (SPEI) apparatus with the second radiation so as to generate second surface plasmon enhanced radiation; and exposing a photoresist to the first surface plasmon enhanced radiation and the second surface plasmon enhanced radiation.

These and other objects, features and advantages of the present disclosure may be better understood by considering the following detailed description of specific embodiments of the disclosure. In the course of this description, reference will frequently be made to the attached drawings.

### BRIEF DESCRIPTION OF THE DRAWINGS

FIG. 1 is a cross-sectional view of an aperture through a metallic film, the film being substantially thicker than the skin depth within which an optically induced electronic excitation occurs, and the aperture having a diameter less than the wavelength of the incident light.

FIG. 2 is a view illustrating the approximate size of the oblong-shaped area illuminated by the light transmitted through the aperture in the film shown in FIG. 1.

FIG. 3 is a graph illustrating the illumination intensity in the illuminated area taken along the line 3-3 of FIG. 2.

FIG. 4 is a cross-sectional view of a thin metallic film that covers a non-metallic substrate material with an aperture through both the metal film and substrate having a diameter less than the wavelength of the incident light.

FIG. 5 is a view illustrating the approximate size of the circular area illuminated by the light transmitted through the aperture in structure shown in FIG. 4.

FIG. 6 is a graph illustrating the illumination intensity in the illuminated area taken along the line 6-6 of FIG. 5.

FIG. 7 is a cross-sectional view of a thin metallic film that covers the surface of a non-metallic substrate material as well as the sidewalls of an aperture through the substrate with the aperture having a diameter less than the wavelength of the incident light.

FIG. 8 is a view illustrating the approximate size of the circular area illuminated by the light transmitted through the aperture in the structure shown in FIG. 7.

FIG. 9 is a graph illustrating the illumination intensity in the illuminated area taken along the line 9-9 of FIG. 8.

FIG. 10 is a cross-sectional view of a thin metallic film which covers a non-metallic substrate material, an aperture through the substrate, and a thin, annular metallic ring surrounding the aperture on the opposing surface of the substrate, with the aperture having a diameter less than the wavelength of the incident light.

FIG. 11 is a view illustrating the approximate size of the circular area illuminated by the light transmitted through the aperture in the structure shown in FIG. 10.

FIG. 12 is a graph illustrating the illumination intensity in the illuminated area taken along the line 11-11 of FIG. 10.

FIG. 13 is a cross-sectional view of a hole structure in which a thin metallic film which covers both surfaces of a non-metallic substrate material, and an annular notch is cut into the film at the exit surface which surrounds and is spaced from the hole.

FIG. 14 is a view illustrating the approximate size of the circular area illuminated by the light transmitted through the aperture in the structure shown in FIG. 13.

FIG. 15 is a graph illustrating the illumination intensity in the illuminated area taken along the line 15-15 of FIG. 14.

FIG. 16 is an end plan view of a multi-aperture probe constructed in accordance with the disclosure.

FIG. 17 is a cross sectional view of the probe seen in FIG. 16 take along the line 17-17.

FIG. 18 is an end plan view of an alternative structure for the multi-aperture probe constructed in accordance with the disclosure.

FIG. 19 is a cross sectional view of the probe seen in FIG. 18 taken along the line 19-19.

FIG. 20 is a cross sectional view of an alternative light barrier structure employing “good” and “bad” metal layers.

FIG. 21 is a schematic diagram of a data storage device that uses an array of nanometric holes to illuminate a data storage array as contemplated by the disclosure.

FIG. 21A is a schematic diagram of an arrangement for reading data from a data storage medium.

FIG. 21B is a schematic diagram of a further embodiment of a data storage system employing the disclosure.

FIG. 21C is a cross-sectional view of a sub wavelength hole array, the storage medium, and the CCD detection array used to implement an optical storage device.

FIG. 22 is a schematic diagram of a Surface Plasmon Enhanced Microscope (SPEM) which embodies the disclosure.

FIGS. 23-25 are plan views of the location of surface patterns surrounding a central aperture used to enhance the illumination from the central aperture.

FIG. 26 is a schematic illustration of a method for varying the in-plane wave vector of photons incident on the illuminated surface of a surface plasmon enhanced illumination device by varying the angle of incidence of monochromatic light.

FIG. 27 is a perspective drawing of one embodiment of a maskless, direct write, photolithography mechanism for translating an SPEI device relative to a photoresist.

FIG. 28 is a schematic depiction of a single channel of a direct write photolithography system.

FIG. 29 is a perspective view of a multi-channel photolithography system employing an array of SPEI light sources fabricated on a rigid substrate.

FIG. 30 is a cross-sectional view of a single channel direct-write device in which the light source is implemented as a separate vertical cavity surface emitting laser (VCSEL).

FIG. 31 is a cross-sectional view of a single channel direct-write device in which a lenslet array is used to focuses light from a surface emitting laser onto the SPEI device.

### DETAILED DESCRIPTION

As described in U.S. Pat. Nos. 5,973,316 and 6,052,238 issued to Ebbesen et al., enhanced light transmission occurs through an array of apertures in a metal film due to the surface plasmons induced in the conductive film by the incident light.

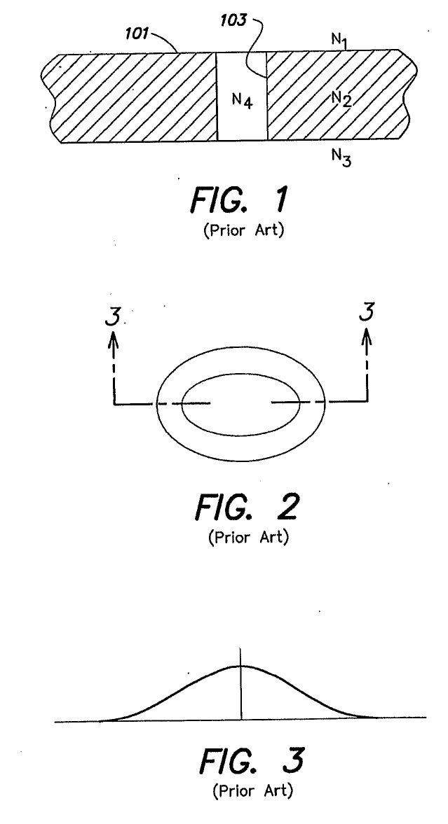

FIG. 1 shows a cross section of an optically thick metal film 101. The term “optically thick” means that the thickness of the film 101 is greater than two times the skin depth. For all essential purposes, this means that there is no direct coupling of the surface plasmons (coherent collective excitations of electrons) at the upper surface (the interface between media of index N₁ and N₂) and the lower surface (the interface between media of index N₃ and N₂) In a typical case, the indices N₁, N₃, and N₄ are equal while N₂, the index of the metal film 101, is substantially different and the metal film 101, unlike the surrounding material, is a conductor of electronic charges.

If the array spacing and the dielectric functions and thickness of the metals and substrates are tailored to attain a high transmission, a significantly higher power density than that transmitted through the single aperture probe used in NSOM (a ratio of about one million per aperture for a 50 nm holes) can be delivered through the apertures. This substantially increases the signal to noise ratio of surface plasmon enhanced microscopy (SPEM) over the NSOM at normal resolutions and is allows a smaller hole size to be used, providing better resolution and dramatically decreasing the dwell time required for an adequate signal to be received.

Unfortunately, the coupling (indirect or direct) between the surfaces of the film 101 seen in FIG. 1 have effects that adversely affect desired resolution. Sönnichsen et al., “Launching surface plasmons into nanoholes in metal films”, App. Phys. Lett. 76, 140-142 (2000) show that, when gold, silver or aluminum films are struck with plane polarized light, surface plasmons are induced in the direction of the polarization. When the plasmons encounter a hole, the coupling to the other side results in light emitted in a prolate shape of a major dimension of about an order of magnitude larger than the hole size. The prolate shape is caused by the radiative decay of the surface plasmons and is a function of the dielectric function of the metal and the wavelength of the incident light and if significant surface roughness exists, the distance between the elements of roughness on that plane.

With a simple isotropic periodically perforated metal film, two potential problems are encountered. First, for use in a microscope and other applications (e.g. optical data storage and photolithography) where small sources of light (high resolution) are required, the existence of the associated prolate pattern diminishes resolution in one dimension severely. Second, the array spacing would have to be such that patterns did not interfere or overlap. Achieving the appropriate spacing would in turn cause the wavelengths at which the surface plasmons are resonant to be shifted, resulting in resonant wavelengths of lower energy. For the excitation of commonly available fluorophores, multi-photon (probably three or four) excitation would be required. Of course, the prolate pattern could simply be accepted and the resolution in the direction of the polarization (along the major axis of the pattern) would default to that dictated by the Rayleigh criterion for that wavelength and numerical aperture.

If a smaller spot illumination size (a nanometric light source) is required, the prolate shape generated from the geometry shown in FIG. 1 is undesirable. If the incident light is polarized, the long dimension of the pattern shape is probably only loosely dependent on the hole size and more dependent on the surface roughness, since rougher surfaces act as very small antennae, which cause SPs to decay, spatially, more rapidly than would be the case if the film surface were smooth. Moreover, the frequency of the light will also affect the pattern shape. Note also that one desirable shape of the intensity pattern for spot illumination should exhibit a step function rather than the extended somewhat gaussian pattern that is seen along the major axis of the prolate shape.

In accordance with the present disclosure, novel structures are used to minimize or eliminate the prolate pattern described above. If the emitting surface (bottom) is no longer continuous but is instead constructed to constrain the propagation of surface plasmons to the immediate vicinity of the aperture, the size of the resulting area of illumination is significantly reduced. If the illuminated surface (top) is left as a continuous conductor with an array of circular holes in it and the bottom is segmented as described above, a photonic funnel can be created. To minimize the effective broadening of the holes due to surface plasmons on the bottom plane, it may be desirable to create a very sharp edge at this point in either a conducting wall or in an insulator with less available charge to minimize any surface-plasmons/photon interaction. It is important to note that the insulator (in the case of a semiconductor) should have a band gap significantly larger than the frequency of the photons, which will be propagating through it.

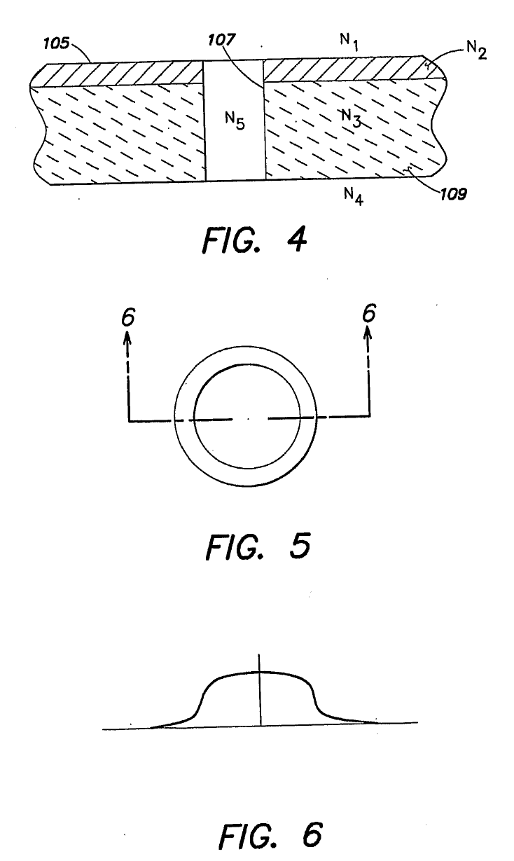

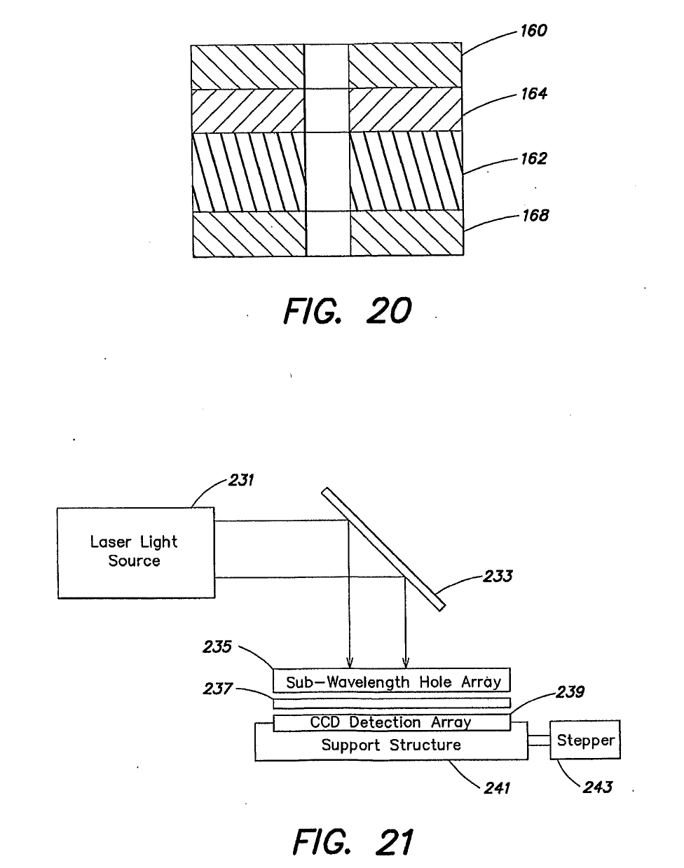

A first improved geometry for the hole array that produces a smaller illumination pattern is shown in FIG. 4 of the drawings. A thin metal conductive film 106 exhibiting the index N₂ is affixed to a substrate 109 constructed of a dielectric material having the index N₃ and a bandgap that is larger than the frequency of the illumination of light. The dielectric substrate 109 can be constructed of a material that is transparent (but need not be) to light at the frequency employed, such as quartz or glass. Note that the aperture 107 need not go through the dielectric substrate if it is transparent, and such a structure may be easier to fabricate. The substrate should have a small index of refraction N₃ compared to the index of the metal N₂. Note also, as discussed later in connection with FIG. 20, that a “bad metal” having poor conductivity at these frequencies (such as tungsten) may be used in place of the dielectric 109 in combination with a “good metal” illuminated layer (such as aluminum). In fluorescence studies, if multi-photon excitement is employed, the bandgap should be larger than the sum of the photonic energies of the photons that would be simultaneously absorbed by the fluorophore. The thin layer of conducting material 105 should be thicker than the skin depth of the metal at the chosen wavelengths. The geometry and composition of the heterogeneous structure seen in FIG. 4 should be chosen so that a maximum of transmission of illumination occurs through the hole 107 at the chosen illumination wavelength. A tunable or broad band light source may also be used to tune the wavelength to predetermined hole dimensions.

The advantage of the geometry shown in FIG. 4 over that presented in FIG. 1 results from the fact that there is no coupling of plasmons from the upper surface of the film 105 to the lower surface of the dielectric material 109. This reduced coupling creates a smaller and more defined illumination pattern with steeper side slopes as illustrated in FIGS. 5 and 6. It is unclear, though, what happens to the energy at the corner interface of the hole 107, the metal film 105 and the dielectric substrate 109, that is, at the boundary of the materials having the indices N₅, N₂ and N₃. If N₁, N₄ and N₅ are not all substantially equal to one (1.0), combinations of differing indices could be used to tailor the transmission of the array apertures for a specific wavelength or method of illuminating the structure. For example, N₁ could be the index associated with an optical fiber, which would be coupled to a remote light source.

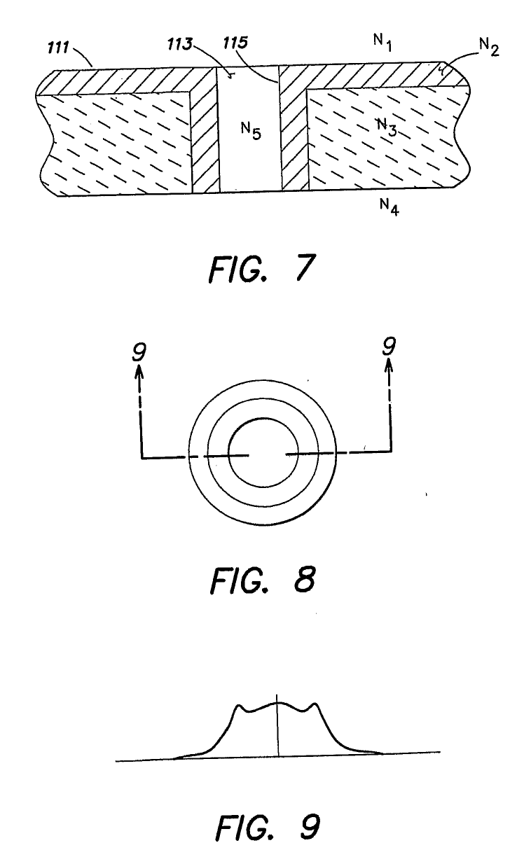

A second hole array structure for reducing the size and increasing the density of the spot illumination is shown in FIG. 7. As before, the structure of FIG. 7 presents at its upper surface a continuous conducting thin film metallic film 111 having the index N₂. The structure differs from that shown in FIG. 4 in that the metallic coating is continued into the interior of the hole 113 as seen at 115. If the thickness of metal layer 115 in the hole interior were greater than skin depth, the effects seen in optically thick metal films as shown in FIG. 1 would be duplicated from the standpoint of optical transmission through the holes. However, a smaller and more concentrated output light pattern is achieved by limiting the propagation length of SPs at the exit surface to the thickness of the film in the hole. Limiting the size of the excited surface area surrounding the hole exit produces a concentrated, circular light pattern as seen in FIG. 8 rather than prolate pattern seen in FIG. 3, thus limiting the size of the light source in only one of its two dimensions. As is the case with the structure shown in FIG. 4, the indices N₁, N₄ and N₅ may be equivalent to 1 in the simplest configuration but other combinations be used to tune the holes for a specific resonance. FIG. 9 graphs the steeply skirted intensity distribution expected across the circular light pattern along the line 9-9 of FIG. 8.

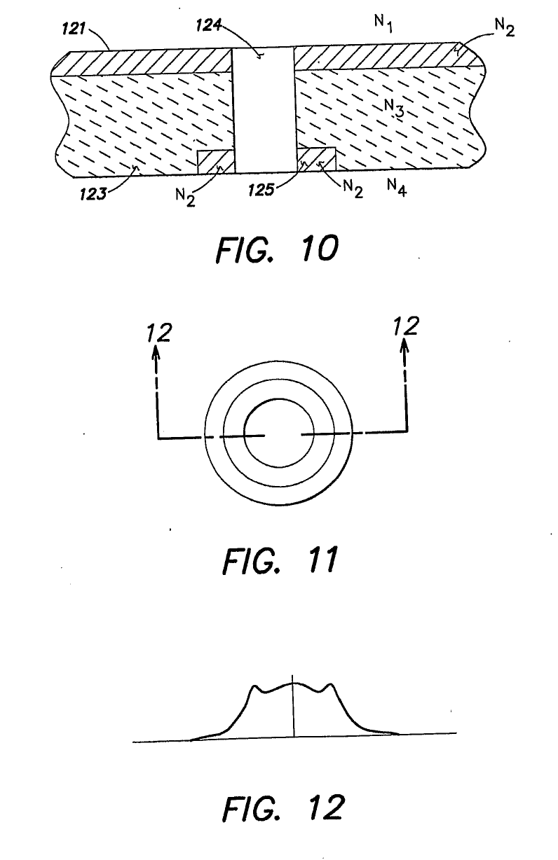

A third structure that may be used as a source of concentrated light is shown in FIG. 10. As in the structures shown in FIGS. 4 and 7, a thin metallic film 121 covers the upper surface of a dielectric substrate 123. A hole 124 through the film 121 and the substrate 123 is not lined with a conductor as in FIG. 7. Instead, an annular ring 125 of conductive material surrounds the exit end of hole 124 at the lower surface of the substrate 123. The conductive ring 125 increases the coupling with the film 124 to improve light transmission through the hole 124 but does not permit the surface excitations surrounding the hole exit to spread beyond the outer periphery of the ring 125, thereby again achieving the more concentrated, steep skirted output light pattern shown in FIGS. 11 and 12.

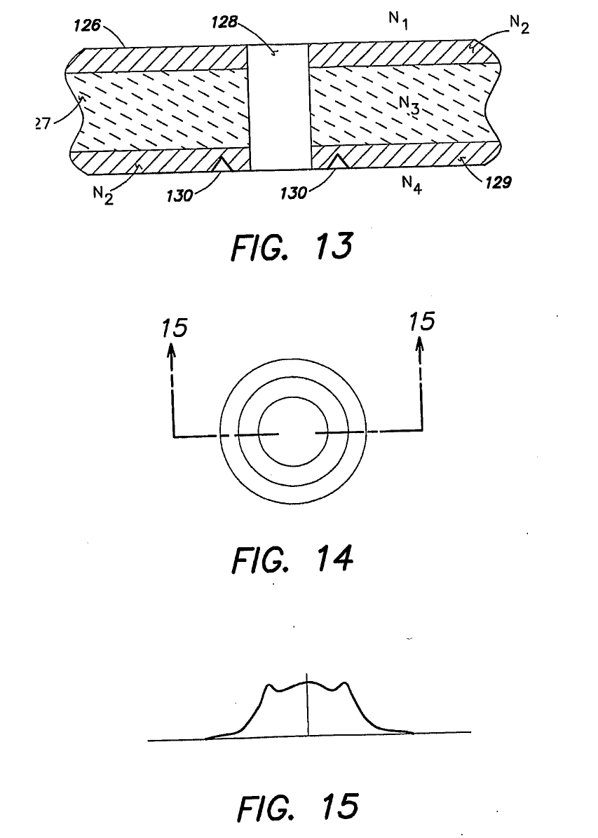

FIG. 13 shows still another structure in which a dielectric substrate 127 is coated on its upper surface with a metallic film 126 and on its lower surface with a metallic film 129. The hole 128 passes through both films and through the substrate and its side walls are not coated. An annular groove seen at 130 is formed in the film 129 and surrounds and is spaced from the hole 128. The groove has a nominal outside diameter of 25 nm and inside diameter of 20 nm. The depth of the groove must be at substantially deeper than the skin depth of the material, i.e., deep enough to act as insulator with respect to induced surface excitations. The groove may have any convenient shape and may be rectangular or triangular as well as semi-circular. Note that, by using a groove of the type shown in FIG. 13, an optically thick metallic structure may be used instead of a dielectric substrate, so that the hole is effectively lined by a conductor. In both cases, the groove serves to contain the coupled electron excitation within a surface area close to the hole exit, thereby preventing unwanted spreading of the illumination pattern. The illumination pattern produced by the hole and groove configuration of FIG. 13 is depicted in FIGS. 14 and 15.

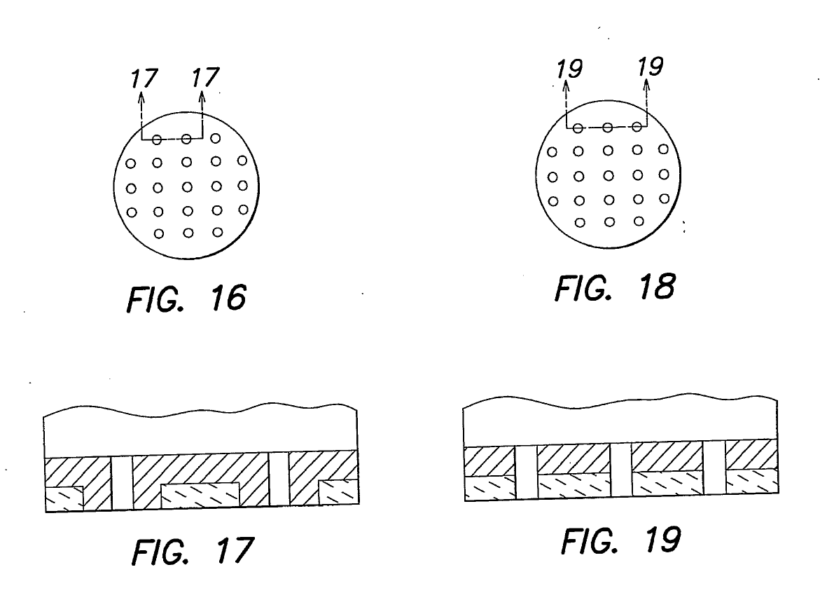

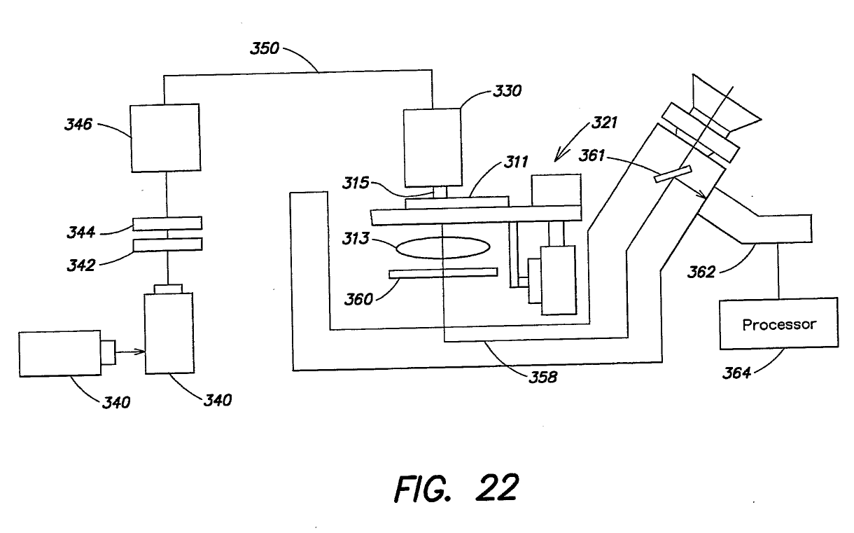

As will be discussed later in conjunction with FIG. 22, the principles of the disclosure may be used to construct a multi-aperture probe (MAP) which may be used to advantage in scanning microscope. FIGS. 16 and 17 illustrate a MAP structure using holes with electrically conducting sidewalls of the type discussed earlier in connection with FIGS. 7 and 13, while FIGS. 18 and 19 show the construction of a MAP having holes whose sidewalls are in part non-conducting as previously discussed in connection with FIGS. 4 and 10 of the drawings.

As also discussed above, another approach to eliminating the prolate pattern is to align the polarization with a slit. If the material through which the photons are propagating has low charge availability (as in slit), there can be very few or no surface plasmons. Also, the propagation of light is supported along the slit and throughput should be higher for an array of slits versus an array of circular holes of the same area. Work done on slits much smaller than the transmitted wavelength (32 nm slit) [see Astilean, Lalarne and Palamaru “Light transmission through metallic channels much smaller than the wavelength” Optics Communications 175 265-273 March 2000] in optically thick metal films shows peaks in the NIR and visible transmission versus incident wavelength curves with maxima in the order of 80% efficiency for the plate with a grid spacing of 900 nm. For the strongest peak, 1.183 μm, this is an extraordinary amount in that almost 10 times the amount of light impinging on the slits is transmitted through them. Also reported are slits of 1 nm widths, which when excited at resonance, achieve 10% efficiency. Astilean et al. conclude that the resonance condition is not only a function of the SP resonance but that the metallic wall linings of the slits act as Fabry-Pérot cavities and that greatly enhanced transmissions occur when the slit satisfies the Fabry-Pérot resonance condition [see Born, M. and Wolf, E. Principles of Optics. Cambridge University Press 6^{th} ed. 1980 p. 326] with an effective index of refraction which depends strongly on the slit width and material.

FIG. 20 shows still another configuration which utilizes the principles of the present disclosure. In this arrangement, the light barrier is composed of three different materials: a “good” metal layer 160 over a substrate consisting of an insulator 162 sandwiched between two layers of “bad metal” 164 and 168. As with the other structures, the “good” metal used in layer 160 is one in which the surface plasmons will decay over a relatively long distance as determined by the surface roughness of the film 160 (which includes the holes) and the relative values of the real and imaginary parts of the dielectric function of film 160 (where a small imaginary part provides a long delay decay length). In contrast, the “bad” metal used in the layers 164 and 168 has a dielectric function with a large imaginary part so that the surface plasmons decay more quickly over a relatively short decay length.

The “bad” metal used in layers 164 and 168 preferably exhibits two additional properties which make a significant contribution to the creation of nanometric light sources. First, the “bad metal” should be opaque to the light emitted from the surface of the “good” metal in thin films. Second, the resonance of the “bad” metal layer(s) should be should be very different than that of the “good” metal. The resonance of the metal layers is determined only by the real part of the dielectric function for metal, the surface roughness of the metal layers, and the dielectric functions of the materials on either side of the metal layer.

The insulator 162 ensures that there can be no surface plasmon communication from top to bottom through bulk plasmons or any other direct electronic interaction. Note, however, that the presence of the insulator 162 may not required if the bad metal satisfies the criteria expressed above; that is, is opaque to light emitted from the good metal layer and has a resonance that is very different from the good metal layer.

For the all of the structures described in connection with FIGS. 4-20, the diameter of the hole should be between about 2 nm and 50 nm. The metallic film layers should, as noted earlier, be at least skin depth of the electronic excitation and may be formed, for example, from gold, silver, aluminum, beryllium, rhenium, osmium, potassium, rubidium, cesium, rhenium oxide, tungsten oxide, copper or titanium (if employed at the appropriate frequencies). Suitable dielectric and “bad metal” substrate materials include germanium, silicon dioxide, silicon nitride, alumina, chromia, some forms of carbon and many other materials including some of the metals listed as “good metals” as the appropriate frequencies. The aperture array with sub-wavelength holes may be fabricated using available focused ion beam (FIB) milling techniques.

The physical structures for producing very small spot and slit illumination may be used to advantage in a number of different applications as next described.

Optical Data Storage Using Small Spot Illumination

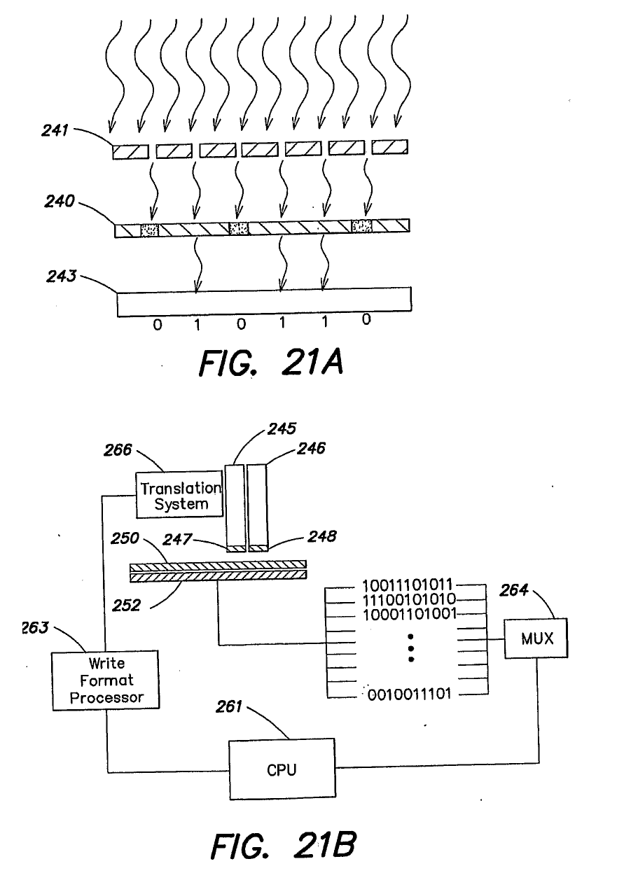

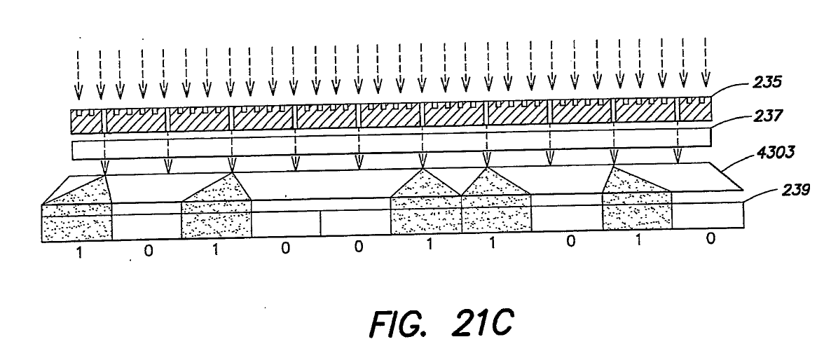

FIG. 21 illustrates the manner in which a nanometric light source array of the type contemplated by the disclosure may be used to increase the storage density in an optical storage device. The optical memory consists of a light source 231, such a solid state NIR laser as shown in FIG. 21. The light from the source 231 is directed onto the metallic film surface of a nanometric hole array 235 using a fold mirror 233. The nanometric hole array 235 collects and funnels the light such that an array of discrete areas of illumination are directed toward the optical storage medium 237. At each area of illumination, a data value stored at that location in the storage medium controls the intensity of the light which passes to a pixel location on a charge coupled device array (CCD) 239 and hence controls the output data value from that CCD pixel. The holes in the array 239, the data storage regions in the medium 237, and the pixel locations in the CCD 239 are equally spaced so that they are properly aligned. A translation mechanism effects movement of the storage medium relative to the hole array in incremental steps, with each step distance being equal to the aperture size.

In the year 2000, commercially available CCD arrays have pixel sizes no smaller than (4 μm)². If this is a limiting case, optics between the storage medium and the CCD array could be used to allow less movement. The step size would then be down to that demanded by the Rayleigh criterion.

Note also that the amount of data stored at each pixel location may be increased by storing more than two signal levels; for example, gray scale or color values may be stored as analog signal magnitudes at each storage location.

The data reading technique employed in the optical data storage system is illustrated in FIG. 21A. The optical medium 240 is illuminated by the spot illumination from the SPEI array 241 and the light transmission through the medium 240 is read by the radiation detector 243 which may take the form of a charge coupled device (CCD) array, a complementary metal oxide semiconductor (CMOS) array, or other array of radiation sensing elements which senses the previously written state of the optical storage medium at each pixel location.

An alternative optical data storage system using SPEI is shown in FIG. 21B. The system employs semiconductor lasers seen at 245 and 246. The laser 245 is fitted with a write mask 247 and the laser 246 is fitted with a read mask. Both masks are SPEI arrays that provide approximately 10,000 apertures each 10-50 nanometers in diameter. The optical medium seen at 250 rotates or otherwise moves with respect to the CCD or CMOS detector array seen at 252. The detector array 252 may be a 100×100 read array, or larger, to provide fast data access. Operating under the control of a CPU 261, a write format processor 263 accepts data to be stored and drives a translation system 266 which moves the write head comprising the laser 245 and the write mask 247. When the data is read from the storage unit, it is collected in parallel by the detector array 252, multiplexed at 264 and returned to the CPU 261.

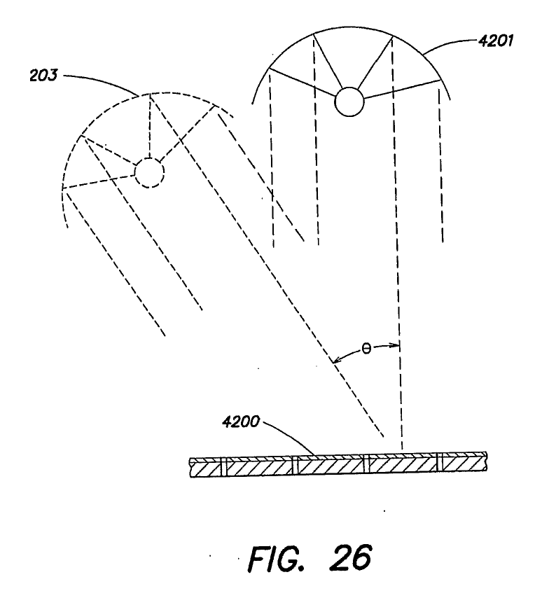

To achieve a rugged, compact system, the SPEI mask (247 or 248) may be fabricated onto the semiconductor or LED light source (245 or 246). The write head (laser 245 and mask 247) may be performed in parallel, but at a different level of parallelism as is achieved in reading. It requires a higher illumination intensity to write data into the optical medium 250 than to read previously stored data due to the need to produce the photochemical change required for writing at an adequate rate. To achieve that increased intensity, the SPEI is modified in the manner discussed below in connection with FIGS. 24, 25 and 26. For writing all, only selected central apertures pass through the SPEI array. At positions surrounding each central aperture, areas of surface roughness (dimples) deeper than the skin depth of the good metal are positioned as shown in FIGS. 24 and 26, or the central aperture is surrounded by an annular groove or raised ring as shown in FIG. 25. This technique allows the extraordinary transmission to be retained while only providing emission from the central aperture. This central aperture then becomes the scanned element that is used to write to the medium. This writing feature can also be used for reading.

Two factors determine the data packing density that can be attained using SPEI data storage: the size of the apertures and the light transmission fraction achieved. Cylindrical holes produced using a focused ion beam (FIB) are typically limited to an aspect ratio of 5-6:1 for the depth versus diameter. Accordingly, for a read or write mask having a thickness is 275 nm, the minimum aperture diameter is approximately 55 nm. By using thinner Si₃N₄ membranes and pushing the limits of the FIB, the ultimate limit is believed to be in the vicinity of 10 nm. Devices have been fabricated on 150 nm thick silicon nitride membranes. For smaller apertures, still thinner membranes may be substituted, or the membrane completely may be completely eliminated. Moreover, the holes need not be cylindrical and may be tapered and still provide high light transmission.

The light transmission fraction is expected to be proportional to the aperture diameter to the first power. “Shutters” may be placed between the light source (the laser 245) and the SPEI device (the write mask 247) to provide parallelism for the write function. The minimum shutter size may limit the density of emitting apertures. The emission from selected portions of the SPEI device may be performed using an LCD (not shown (to block the light, or the local dielectric function at the interface may be alerted as demonstrated by Kim et al. in the paper “Control of optical transmission through metals perforated with sub wavelength hole arrays,” Opt. Lett. 24, 256-258 (1999). In still another shuttering method, conductive wires may be attached to influence the individual resonant patterns in the device and, thereby, alter the electron density and the resonance of the surface plasmons in the area local to selected aperture in question, thereby modulating the aperture's emission pattern.

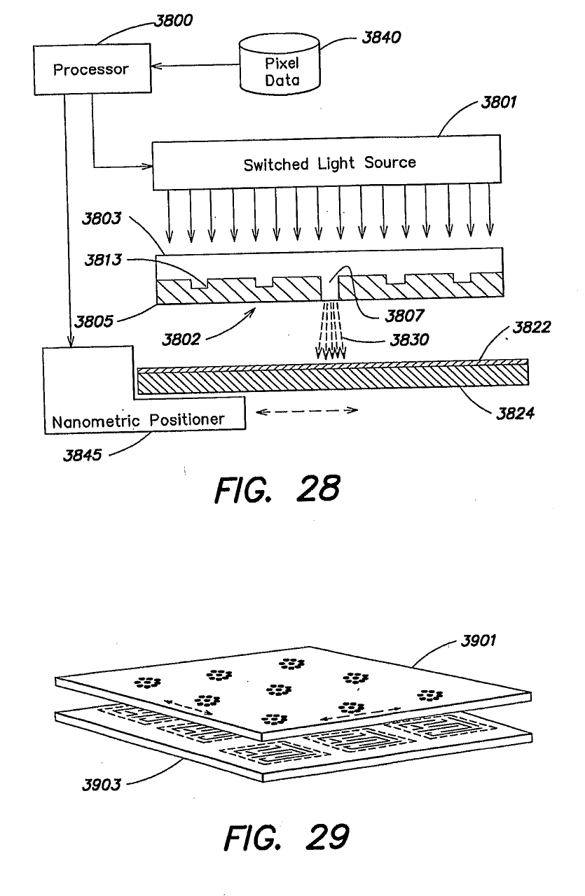

In still another arrangement, each SPEI light funnel in the array 235 may be independently illuminated by a solid state light source, such as an LED or a VCSEL as described later in connection with FIGS. 28-31. As discussed below, VCSEL arrays are widely used in optical data transmission systems and are capable of switching speeds of 1 GHz.

The optical storage media 237 can take a number of conventional forms which are known in the art. An optical storage medium typically responds to write illumination by changing its physical characteristics, such as a change in its emissivity, in its reflectivity, or in its fluorescence. Examples of existing optical storage media include the following:

U.S. Pat. No. 4,239,338 issued to Borrelli et al. describes an optical data storage film that exhibits both high levels of induced birefringence and relatively high transmittance at near infrared light wavelengths and uses optical bleaching with polarized light to induce dichroism and birefringence in silver-containing silver halide photographic emulsions.

U.S. Pat. No. 5,188,923 issued to Ahn et al. describes an optical storage medium composed of a substrate consisting of a transparent dielectric material such as a polymer upon which a vacuum deposited discontinuous film of nucleated metallic islands is deposited having thicknesses of less than about 100 Angstroms. The islands are separated by a short distance for facilitating coalescence of particles together to effect writing of spots at low energy by the lateral motion and gathering of the islands during heating of the islands by an illumination beam.

The choice of the media for SPEI based high-density data storage is driven by the physics of the technology. In order to take advantage of parallel read/write architecture of the SPEI array 235 to produce very high density data storage, the resulting bit size on the storage media 237 will be well below the wavelength of the illumination; that is, 50 nm or less. However, light cannot be transmitted efficiently through storage media bit locations of this size. This is not an issue for write events, but is a major concern for read events. Therefore, a means of generating a readable signal upon bit interrogation must be employed. One possible approach is to create a point source of light that is turned on by illumination from the read illuminant. This may be accomplished by utilizing the phenomenon of fluorescence.

For example, phase change technology, in which heat generated by the nanometric light source 235 write data on the recording surface of optical media by altering the reflective quality of the recording surface. This is done by changing the physical state of the media's recording layer from a crystalline to an amorphous state, which produces bright to dark marks on the media. The same light source 235, set at a lower intensity, may then be used to read the media. This “write once” technique prevents data from being overwritten or altered.

FIG. 21C shows the sub wavelength hole array 235, the storage medium 237, and the CCD detection array 239 in more detail. When the storage medium takes the form of a fluorescent material, two different modes of operation can be used. A material that is inherently non-fluorescent, but which becomes fluorescent when sufficiently illuminated, may be used so that a write event turns on fluorescence in selected bit locations. The second mode uses a material that is inherently fluorescent and the write event turns off the fluorescence in selected bit location. The fact that fluorescence occurs on the nanosecond time scale makes it well suited for this technology.

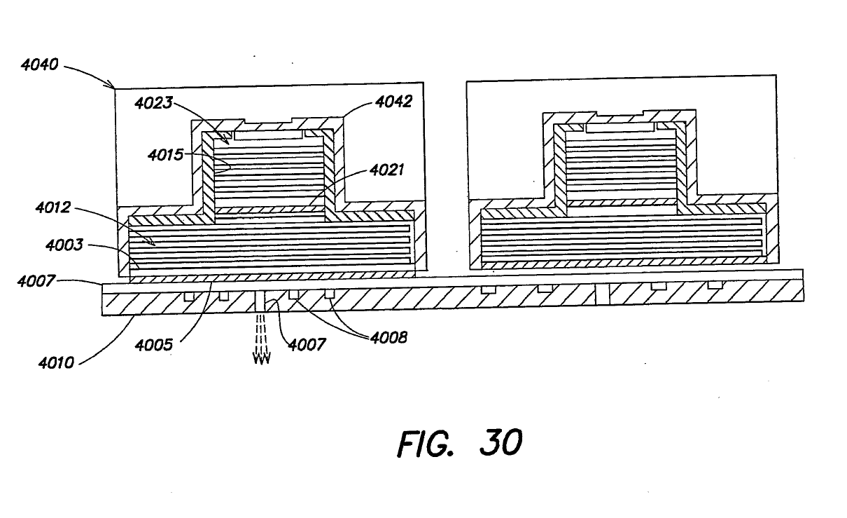

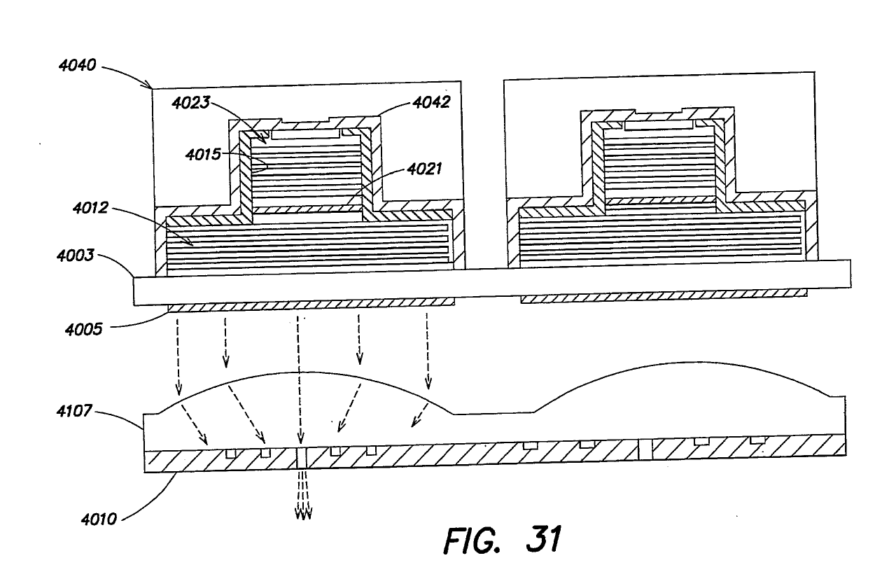

A three-level illumination source (such as the controlled VCSEL lasers described later in connection with FIGS. 30 and 31), may be used to selectively illuminate individual SPEI light funnels in the array 235. At the highest level of illumination, the light striking the storage media 237 alters the materials fluorescence at that bit location. On a read event, all of the SPEI funnels are illuminated with a reduced light level (insufficient to alter the properties of the material, but sufficient to cause those locations for which fluorescence has been left in the ON state by the prior write operation to fluoresce. This fluorescent radiation is spread by the beam expander 4303 to better illuminate the active area of each CCD cell in the detection array 239 which reproduces the stored data.

There is a multitude of fluorescent species that could be considered as the active-component of the media. Each has unique excitation and emission characteristics. By controlling the amount of energy (light) a bit is exposed to during a write event, the amount of fluorescence either created or destroyed can be controlled, thereby allowing the creation of multiple fluorescence levels. The amount of energy available during a write event is more than sufficient to perform chemistry on the media at short time scales (nanosecond or less). Using physical modeling, the energy available on the ‘write’ medium for a SPEI test configuration with a 50 nm aperture and assuming a conservative doubling of the incident intensity though the plasmon assisted “funneling of light” at an 11 megabits/sec data rate is estimated be 3×10⁻¹² joules on each ‘write’ medium. Using physical property data for typical polymers, this level of energy has been shown to be capable of producing a local temperature rise of more than 600 C. for a ‘write’ event.

Fluorescence occurs when a molecule is excited by light energy and then releases this excess energy in the form of light. Molecules tend to primarily lose this excess energy by mechanical means such as vibration and collision with other molecules. However, some molecules (especially rigid aromatics) have a marked tendency to release excess energy through a photon. This can be through fluorescence (singlet to singlet transition) or phosphorescence (triplet to singlet conversion). Fluorescence is a significantly more common pathway and is also much better suited to the task at hand due to the long time constants of phosphorescence (up to seconds).

The fluorescence of a specific chemical entity occurs with characteristic excitation and emission spectra. Because of the multiple vibrational levels available in both the ground and excited states, the excitation and emission bands are relatively broad. The wavelength of emitted light is always of longer wavelength (lower energy) than the excitation wavelength. The frequency shift is usually on the order of 50 to 100 nm, making it easy to filter the excitation light from the emission. It has been shown that a fluorescent species could be incorporated into a thin polymeric film and that the fluorescence could be destroyed in specific areas of the film at size scales of approximately 100 nm. A poly(methylmethacrylate) (PMMA) solution containing TRITC (a rhodamine based fluorescent tag for proteins) was spun cast onto a glass plate to a thickness of 200 nm after solvent removal. This coating was etched by FIB (focused ion beam) milling to create now fluorescent features on a size scale of approximately 100 nm.

Coumarin laser dyes are the preferred fluorescent species in the media. These dyes have high fluorescent efficiencies with appropriate excitation and emission spectra. Many of the coumarins absorb light at approximately 400 nm and emit at 500 nm. In addition, these dyes are known to be susceptible to photo-oxidation; when they are hit by light of the appropriate wavelength and intensity, they irreversibly lose their fluorescent character. Coumarin 102 dye has particularly desirable properties in this application. Several coumarins were checked for compatibility with poly (methylmethacrylate) (PMMA) solutions and were found to be sufficiently soluble to introduce appropriate quantities into cast films.

The use of SPEI to implement optical data storage systems possesses numerous important advantages. Using the techniques described above, it is believed that data storage devices capable of storing 2.8 Terabit/in² (with 10 nm apertures and with the data stored in a binary format) can be fabricated. SPEI arrays with 50 nm (82 Gigabits/in²) apertures have been constructed, and aperture sizes as small as 2 nm are possible to potentially yielding 70 Tb/in² storage densities. As noted earlier, Gray scale or color recording offers the potential for further increases in data density. Data may be read from the device in massively parallel format, achieving read rates that exceed 1500× those for CD technology. High light transmission fractions (15.3% of the light incident on apertures (50 nm) is transmitted in propagating modes to the optical medium) have been achieved in very early devices of SPIE architecture. Because the light is propagating, sub wavelength illumination may be achieved without resorting to near-field techniques. A wide range of illuminating wavelengths may be employed, ranging from the deep ultraviolet to infrared, which permits the selection of a wavelength to optimize the performance of the photochemical used as the optical storage medium. The high light transmission fraction combined with flexibility in the wavelength of the light delivered provide the photochemist with the possibility of either using existing chemistries or creating new formulations to take advantage of the properties of light emitted from SPEI devices. The system operates in ambient environments (no cryogenic temperatures or vacuum are required for operation). SPEI data storage is compatible with a broad range of applications to meet the needs of large data centers, high density backup system, and storage for desktop and handheld devices. Unlike magnetic technologies, data stored in a SPEI medium is immune to electromagnetic impulse.

Surface Plasmon Enhanced Microscopy

FIG. 22 of the drawings illustrates the use of the nanosecond light source array as contemplated by the disclosure to construct a “Surface Plasmon Enhanced Microscope” SPEM). A sample 311 is placed between the objective lens 313 of the microscope and the multi-aperture probe (MAP) 315. The sample is mounted on a transparent, flat substrate placed on a translation stage 321 capable of nanometric movement. The MAP 315 is then moved into close proximity to the sample 311 and held in place by a compressive force module or proximity sensor 330. In fluorescence mode, light is emitted by a light source, such as a pumped laser, a light emitting diode, an arc lamp or other white light generator, 340 and transmitted via neutral density filters 342, polarizers 344, a fiber coupler 346 and an optical fiber 350 down to its terminus at the MAP 315, where it is emitted through an array of holes in a mask that has been fabricated onto the end of the optical fiber. The light leaving the holes strikes the sample 311 at its surface. The far field light path 358 from the objective 313 passes through a low pass filter 360 to a beam splitter or mirrored shutter at 361 which redirects the light to a array charge coupled device (CCD) 362 that converts the light into electrical signals which are passed to the processor 364 which performs image capture (frame grabbing) and other image processing functions.

In fluorescence mode, the impinging light is absorbed by fluorophores, which resonate, emitting photons at a different frequency. The fluorescent light is collected in the far field by the objective lens and then transmitted into oculars 370 or to the data collection device (e.g., the CCD array 362.)

Once the entire sample has been illuminated by the array of apertures, the resulting fluorescence is collected in the far-field. The MAP 315 is then raised and the sample 311, or the MAP, is indexed to the next position and another set of measurements is made. This process is repeated until the space between the spots, 250 nm to 600 nm, has been scanned. This is a much easier and faster task than with NSOM. In an alternative arrangement the MAP is simply scanned and the raising and lowering steps are eliminated.

It should be clear from the above discussion that it would be difficult to design a probe of the types above with the aim of efficiently transmitting a multiple of wavelengths chosen to maximize the excitation of a suite of fluorophores. One solution is to make tunable MAPs by dynamically modifying the effective dielectric function of the secondary metal (the metal probably would be replaced by a semiconductor) during operation. By changing the dielectric function of the surface below the primary metal, the frequency of emission can be changed substantially. [See Kim, T. J., Thio, T., Ebbesen, T. W., Grupp, D. E. & Lezec, H. J. Control of optical transmission through metals perforated with subwavelength hole arrays.” Opt. Lett. 24, 256-258 (1999) using a twisted-nematic liquid crystal under an array]. It has also been shown that the application of a magnetic field has strong effects on the dielectric function [see Strelniker, Y. M. & Bergman, D. “Optical transmission through metal films with a subwavelength hole array in the presence of a magnetic field.” Phys. Rev. B 59, 12763-12766 (1999). Another method of tuning the array may be to have domains surrounding the apertures in which the density of electrons can be modified by passing an electric current through that domain. The small capacitance of the domain would affect the density of the electrons and, hence, the resonance of the surface plasmons.

Multiple MAPs could be constructed with parameters tailored to each fluorophore of the chosen suite. Each probe would be interfaced to the sample and would present a roughly monochromatic source. As the widths of the peaks of the resonances of the MAPs will be broad (about 20 nm FWHM), the fluorophores will have to be chosen well with significant distances between their excitement wavelengths. In this case, the SPEM will probably be limited to only a few (maybe 6 or so) different fluorophores. However, the quantum dot offers great promise. Bruchez et al. [“Semiconductor Nanocrystals as Fluorescent Biological Labels” Science 281 1998.] have successfully used quantum dots as biological markers. Importantly, the quantum dots may be excited by a single source and to be multiplexed such that multitudes of dots can be detected and identified simultaneously.

SPEM has been conceived with clinical and basic research applications in mind and the user interactions have been structured to make it an easy technique to use. The basic steps, for both clinical and basic research use, are: 1) Prepare the sample; 2) Select the cells of interest from the slide; 3) SPEM automatically captures the data; 4) Review the results and generate specific database analyses.

Step 1. Prepare the Sample: In the clinical application the only additional sample preparation step required is to add the antibody-label reagent to the slide and incubate. The tissue sample preparation steps currently in use for pathology slides are done prior to adding the SPEM labeling reagents (antibody-fluor complexes). Generally for cell culture samples the cells will be embedded in paraffin and then treated as tissue samples for the purposes of preparing them for analysis in SPEM. It would be possible, though, by using an actively cooled, transparent, thermally conductive substrate, to investigate frozen tissue samples and, under suitable conditions, it should be possible to study live cells using SPEM.

Step 2. Select the cells of interest from the slide: With SPEM the user looks at the slides with a standard far-field microscope prior to the high resolution investigation. This allows the user to make use of the morphology data available today and select cells for farther analysis that are the most interesting. To accomplish this, the SPEM system will incorporate a module that allows the user to digitally mark (record the x-y coordinates) the cells for further analysis. This allows the user to gather data on different cell types, cells at different stages of the cell cycle, and multiple cells of the same type to increase the statistical power of the near-field analysis. This also should allow the user to create multiple slides from the same cell representing sequential cuts from the microtome. The resulting SPEM data can then be reconstructed to create a three dimensional data set of protein locations and expression.

Step 3. SPEM automatically captures the data: The SPEM system will execute the illumination and far-field collection steps described above to generate a database of protein localization and expression information.

Step 4. Review the results and generate specific database analyses: The database created in the previous step provides the user with the ability to create custom queries to address the biological or clinical question under investigation. It is expected that as SPEM matures there would be a library of specific database queries that would be used. In particular, for clinical use pathologists would have a set of standard analyses that are performed with the SPEM to elucidate molecular signatures of cancer.

SPEM generates a data file consisting of the location of every fluor detected in the cell, and the protein with which it is associated. This data file can be analyzed in a number of ways, including:
i) Generating a map of each protein's location within the cell that is superimposed on an image of the cell.
ii) Providing the number of copies of each protein that were detected.
iii) Statistics for a number of conditions:
   (a) Percentage of copies in the nucleus or cytoplasm
   (b) Number of copies of a protein that are within a user specified distance of either another protein, or a cellular feature (e.g. cell membrane)
   (c) Comparisons between cells (e.g. mutant and wild type)
   (d) Comparisons of protein locations and expression levels between cells at different stages of the cell cycle.
   (e) Comparisons between cells at different developmental levels
iv) Assist in the selection of therapies and determination of prognoses for cancer patients based on molecular signatures of cancers.

The strengths of SPEM include:
(1) The ability to obtain protein localization and expression data for multiple proteins in a cell from either cell culture or a tissue sample.
(2) Localization resolution better than 75 nm, and possibly as low as 10 nm.
(3) Protein expression data based on protein levels, not on mRNA.
(4) Permits the study of low copy number proteins.
(5) Less sensitive to vibrations than NSOM and Atomic Force Microscopy. The level of vibration isolation that is needed is similar to standard microscopy techniques.

The MAP used in a SPEM should:
(1) Have an array 75 nm (or smaller) holes that can illuminate a tissue sample with enough energy to excite fluors that have been bound to specific proteins in the sample.
(2) Have a diameter of at least 20 μm in order to cover a typical cell.
(3) Have the holes in the array spaced far enough apart to permit collection of optical data from the fluors using far-field optics (greater than the distance imposed by the Rayleigh criterion for the objective lens being used for collection and the emission wavelength of the lowest frequency fluorophore.)
(4) Maintain high resolution registration of the locations of the holes in the array relative to the far-field optics.
(5) Have optical and thermal conductances that are high enough to avoid deteriorating levels of thermal expansion of the MAP and heating of the sample.

Fabrication of the MAP should be undertaken with the following parameters in mind: the ability to control aperture size (geometry and thickness); the ability to control aperture spacing; the nature of the materials (e.g. purity, continuity); and the characteristics of the coating needed (e.g. continuity and thickness).

In the metal film experiments above, the holes in the films were created by two methods, both achieving excellent cylindrical geometry. In the Sönnichsen experiments, a suspension of polystyrene beads was spin-cast onto a very thin (1 nm) adhesive layer on a glass substrate and a subsequent metal film evaporated onto the adhesive and the spheres. The spheres and the metal covering them were then removed by ultrasonification. In the experiments conducted by NEC Research, the holes were created by focused ion beam milling (FIB). This method allowed more latitude in the hole size and spacing in the metal film.

Because the preferred structures are both heterogeneous and require that the hole spacing is uniform (for scanning purposes) or at least well characterized and repeatable from MAP to MAP, the method of spin casting is not useful. FIB can be used but may be expensive for the use of SPEM in clinical settings. Another proposed method of fabrication is to use a naturally occurring structure of alumina. Alumina can be anodically etched to produce a uniform nanometric, closely packed honeycomb-structure over large areas [see Keller et al. J. Electrochem. Soc. 100 411 1953, Thompson et al. Nature 272 433 1978]. By using micromanipulation, holes could be filled with an insulator or conductor leaving only apertures where desired. The structure would then be coated with the chosen electrical conductor and the bottom surface milled away using FIB.

The SPEM microscope illustrated in FIG. 22 may be implemented using commercially available components. An inverted fluorescence microscope such as a Zeiss IM35 or a model from the Zeiss Axiovert family would be suitable for modification. The microscope should have at minimum, two high numerical aperture (1.3 or greater) Plan-Apochromat objectives; one for high magnification (100×) and one for medium magnification (63×.) Because the exciting photons are traveling in the MAP, and there is no ultraviolet light involved, special glasses and coatings are not required. The above objectives have been corrected at the red, green and blue wavelengths for chromatic aberration and will, hence, not be a problem with different fluorescing colors.

At low levels of fluorescence (low light input is desired to minimize the effects of photobleaching and possibly, with two-photon excitation, stimulated emission depletion) that may be seen in the SPEM, cooling is required when using a charge coupled device (CCD) array to maximize signal to noise ratio. Zeiss manufactures a suitable high resolution (1300×1030 pixels) thermoelectrically cooled CCD array/frame grabber package called Axiocam with color density of 14 bit color classification which is adequate for purposes of multiple fluorescence capture and discrimination. The Axiocam is sold by the Microscope Division of Carl Zeiss with software called Axio Vision that is supplied along with the CCD array, a thermoelectric cooler, frame grabber and image analysis software that are integrated with and designed specifically to mate to the Axio microscopes.

Translation of the sample relative to the MAP and collection optics requires a 3axis translation stage shown generally at 321 in FIG. 22. The step size of the translation stage and its resolution should be less than the required resolution desired of the spatial resolution of fluorophores in the sample. Mad City Labs (Madison, Wis.) offers such a device called the Nanobio350. The controller is delivered with LabView software to make integration with the imaging system easier.

Although the above-noted CCD array is color sensitive and discriminating, it is sensitive into the wavelength regime (NIR) of the emission laser. So that the pixels are not saturated with the stimulating radiation and to avoid more computation than necessary, an optical low pass filter should be placed in the path between the CCD input and the objective lens of the microscope. There are numerous suppliers for such filters. If a laser light source is used, a grating compensation system may need to be employed to avoid the dispersion that would otherwise occur in the fiber. These are available from Coherent.

The current factor that limits the number of proteins that can be simultaneously characterized using SPEM is the limited availability of spectrally distinguishable fluorophores. Many researchers are working on this issue and it is expected that SPEM will benefit greatly from these efforts. Some of the more interesting candidates are described below.

Because the MAP will be designed for efficient transmission of one specific wavelength of light, a set of fluorophores that can all be excited by the same wavelength will need to be selected. There are two promising methods for this: 1) two-photon excitation of fluorescent dyes, using an infrared light source, and 2) quantum dots, using a blue-violet light source. For fluorescent dyes, we would need a set with well-separated emission wavelengths and narrow spectral peaks. At least two vendors offer products that meet these criteria: Molecular Probes of Eugene, Oreg. offers a set of seven BODIPY dyes, and Amersham Pharmacia Biotech (www.apbiotech.com) offers a set of five Cy dyes. In addition, new dyes are introduced frequently. Quantum dots are not yet commercially available for biochemical labeling, but are expected to be in the near future. By tailoring the size of the cavity, quantum dots can be made with any desired emission wavelength, so conceivably more than seven could be used within the visible-light spectrum. However, quantum dots are significantly larger than fluorescent dye molecules, 10-20 nm vs. 1-1.4 nm effective diameter. This makes fluorescent dyes the more attractive option. However, if two-photon excitation overheats the SPEM probe, quantum dots will be used for the multiple-labeling experiments.

Quantum dots are nanometer size semiconductor particles with sub-wavelength size pits grown or machined into them. The dimension of the pit determines the color of light emitted from a quantum dot. The pits have dimensions 2 nm (for green light) to 5 nm (for red light), and the overall particle has a dimension of 10-20 nm. It should be easier to develop new quantum dots with precisely tuned emission wavelengths (compared to developing a new fluorophore) by tailoring the exact dimensions of the pits in the quantum dots. Quantum dots have a narrow spectral peak width, with a full width at half maximum (FWHM) of 30-35 nm [see M. Bruchez Jr., M. Moronne, P. Gin, S. Weiss, and A. P. Alivisatos, “Semiconductor nanocrystals as Fluorescent Biological Labels”, Science, 281, 25 Sep. 1998, p. 2013-2016.]. This is comparable to the seven Molecular Probes BODIPY fluorescent dyes, which have spectral peak widths of 22-35 nm FWHM [FIG. 1.2 of Molecular Probes CD handbook]. Narrow spectral peak widths allow many colors to be distinguished, allowing many reporters to be used simultaneously.

In addition to fluorescent dyes, and quantum dots mentioned above, other types of reporters are also in development. Multiplexing arrangements, which allow a more complex code in each reporter tag, are also in development.

At present, all of these approaches produce tags that are too large. Nanobarcodes (10-20 nm diameter×30 nm long) consist of chips with stripes of reflective gold, silver, and platinum metal. The width and spacing of the lines can be altered. Colloidal particles have been used to tag beads for combinatorial synthesis [see Battersby B J, Bryant D, Meutermans W, Matthews D, Smythe M L, Trau M, Toward Larger Chemical Libraries: “Encoding with Fluorescent Colloids in Combinatorial Chemistry”, Journal of the American Chemical Society, 122: (9) 2138-2139, Mar. 8, 2000]. In this scheme, a 100-micron diameter bead holds multiple 1-micron diameter colloidal particles. Each type of colloidal particle holds a unique combination of fluorescent dyes. PEBBLE (Probe Encapsulated By Biologically Localized Embedding) sensors consist of fluorescent dyes encapsulated in a polymer matrix; these particles can be as small as 20 nm. While these have been used for sensing ion concentrations in cells [see 1 Clark, Heather A; Hoyer, Marion; Philbert, Martin A; Kopelman, Raoul, “Optical Nanosensors for Chemical Analysis inside Single Living Cells. 1. Fabrication, Characterization, and Methods for Intracellular Delivery of PEBBLE Sensors”, Analytical Chemistry, 1999, v. 71, n. 21, pp. 4831-4836; and Clark, Heather A; Kopelman, Raoul; Tjalkens, Ron; Philbert, Martin A, “Optical Nanosensors for Chemical Analysis inside Single living Cells. 2. Sensors for pH and Calcium and the Intracellular Application of PEBBLE Sensors”, Analytical Chemistry, 1999, v. 71; n. 21, pp. 4837-4843], the technique may be extendable to labeling proteins.

It is possible that the light output from the holes in the MAP will cause illumination of fluorophores or quantum dots in planes substantially below the surface over which the MAP sits. These molecules could be excited by the spreading photons and may, therefore, not be directly in line with the axis of the holes but could be in between the axes of several holes resulting in a weak magnitude positive signal at more than one location, yielding incorrect spatial information and possibly concentration or color. Methods to reduce this misinformation could be (but certainly aren't limited) to making the tissue sample or the image sample as thin as possible or using multi photon excitement. Because of the squared dependence of the two photon excitement of location, there will be a substantially higher chance of two photons arriving concurrently directly in line with the axes of the holes than anywhere else below the MAP, potentially enhancing resolution.

Other modifications to the MAP may be implemented to modify the resonant wavelengths. One method would be to change the in-plane magnetic field of the MAP. It has been shown the direction and the magnitude of the field can dramatically affect the resonant wavelengths by affecting the effective dielectric functions of the metals. Another method may be to change the density of electrons in the metals to also affect the effective dielectric functions. This could be achieved in numerous fashions. The simplest would be simply to “pump” electrons into the metal. Possibly, localization of charges and/or magnetic fields could allow the MAP to perform read and write operations in storage media and could be used a polychromatic excitation source for fluorophores.

Single Aperture Resonance Configurations

Any of the structures described above in connection with FIGS. 4-20 can be modified so as to facilitate a resonance condition to generate surface plasmon enhanced radiation from a single aperture. For example, in one such implementation, all but the central aperture in a set of apertures would be changed from apertures that go through the barrier to elements of surface roughness (dimples or protuberances) that are deeper than the surface plasmon skin depth and the same diameter as the aperture. Alternatively, the dimples surrounding the central aperture can be replaced with an annular groove or raised ring having a width equal to the emitting hole diameter and a depth (or height) greater than the surface plasmon-skin-depth. This technique allows the extraordinary transmission to be retained while only providing emission from the central aperture. In one exemplary application according to the present disclosure, this central aperture may be employed to generate a single light beam that is used to write patterns in photoresist to perform lithography.

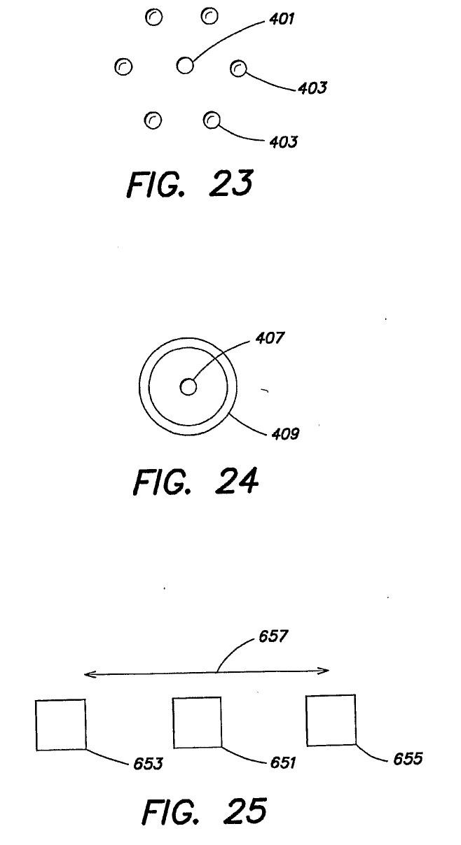

Exemplary structures constituting a resonance configuration comprising a single aperture and one or more features that cause a variation in a dielectric function along a first surface of the metal film proximate to the aperture are illustrated in FIGS. 23, 24 and 25. For these resonance configurations, the aperture and the one or more features are configured so as to cooperatively facilitate a resonance condition for surface plasmon enhanced radiation generated by the SPEI apparatus, based on incident radiation that irradiates the first surface of the metal film.

FIG. 23 illustrates a hexagonal pattern of apertures (one emitting aperture 401 surrounded by six dimples 403), where the relationship between a resonance condition and the spacing of features (i.e., the relationship that establishes the maximum wavelength at which photons in the incident radiation are resonant with surface plasmons in the metal film), assuming normal incidence of the incident radiation to the irradiated plane of the metal surface, is governed generally by the equation:

$${λ}_{max} = {{a}_{o} ( \frac{4}{3} ( {i}^{2} + ij + {j}^{2} ) )}^{- \frac{1}{2}} {( \frac{{ɛ}_{1} {ɛ}_{2}}{{ɛ}_{1} + {ɛ}_{2}} )}^{\frac{1}{2}} , \qquad ( 1 )$$

where λₘₐₓ is the maximum wavelength corresponding to the resonance condition, ∈₁ and ∈₂ are the real portions of the respective dielectric constants for the metal and the surrounding medium through which the incident radiation passes prior to irradiating the metal film, and αₒ is the lattice constant (spacing between dimples/protrusions/apertures). The indices i and j are integers characterizing a particular branch of the surface plasmon dispersion (See Raether, Heinz “Surface Plasmons on Smooth and Rough Surfaces and on Gratings” Springer Tracts in Modern Physics v. 111, Springer-Verlag, Berlin 1988).

Lattice or array patterns other than the hexagonal pattern illustrated in FIG. 24 are permissible according to equations similar to Eq. (1) above, in which the integer indices i and j are modified for the specific lattice type. For example, the maximum wavelength corresponding to the resonance condition in a square array, assuming normal incidence of the incident radiation to the irradiated plane of the metal surface, is governed generally by the equation:

$${λ}_{max} = {{a}_{o} ( {i}^{2} + {j}^{2} )}^{- \frac{1}{2}} {( \frac{{ɛ}_{1} {ɛ}_{2}}{{ɛ}_{1} + {ɛ}_{2}} )}^{\frac{1}{2}} . \qquad ( 2 )$$

If the square array is reduced to a linear array (i.e., a “one-dimensional grating” in which a center aperture is flanked, along a single axis, by dimples or protuberances), the index j in Eq. (2) above would be zero, and the equation reduces to:

$${λ}_{max} = \frac{{a}_{o}}{i} {( \frac{{ɛ}_{1} {ɛ}_{2}}{{ɛ}_{1} + {ɛ}_{2}} )}^{\frac{1}{2}} , \qquad ( 3 )$$

in which the parameter αₒ may be thought of as a grating constant in a one-dimensional implementation (as opposed to a “lattice” constant, as indicated above for the two-dimensional examples). FIG. 26 illustrates an example of such a resonance configuration, including a central aperture 651 flanked by surface irregularities 653 and 655. In one aspect of the resonance configuration shown in FIG. 26, the use of square apertures may be particularly suitable for polarized radiation that irradiates the resonance configuration (indicated by the arrow 657), as the square apertures may significantly reduce variations in the grating constant by ensuring that the aperture-to-aperture spacing is uniform in the direction of radiation polarization 657.

While the configurations discussed above in connection with Eqs. (1) through (3), and exemplified by the hexagonal pattern shown in FIG. 23 and the linear pattern shown in FIG. 25, provide examples of single aperture devices involving a number of periodically arranged features (i.e., a periodic surface topology) proximate to the aperture, other embodiments of the present disclosure relate to resonance configurations involving only a single aperture and a single feature disposed proximate to the aperture. For example, FIG. 24 shows an alternative arrangement for facilitating a resonance condition according to yet another embodiment, in which a single emitting aperture 407 is surrounded by a single annular groove 409. In one aspect, a width of the annular groove is equal to the diameter of the emitting hole. In another aspect, a depth of the single annular groove should be greater than the skin depth of surface plasmons induced by the incident radiation. In yet another aspect, a single raised ring may be employed as an alternative to the single annular groove 409. Using such a resonance configuration in which the aperture 407 is at the center of a single annular groove or single raised ring, the maximum wavelength corresponding to the resonance condition (again assuming normal incidence of the incident radiation to the irradiated plane of the metal surface) is governed generally by the equation:

$${λ}_{max} = \frac{ρ}{i} {( \frac{{ɛ}_{1} {ɛ}_{2}}{{ɛ}_{1} + {ɛ}_{2}} )}^{\frac{1}{2}} , \qquad ( 4 )$$

in which ρ denotes the radius of the annular groove or raised ring from the centrally positioned aperture within the annular groove/raised ring.

The resonance configuration shown in FIG. 24 represents a non-periodic structure that includes only two elements (at least one of which is an aperture) proximate to each other in a metal film. Applicants have recognized and appreciated that a periodic surface topology (as represented by the configuration of FIGS. 23 and 25) is not necessarily required to achieve enhanced transmission through a single aperture. Rather, in other embodiments of the present disclosure (e.g., as illustrated in FIG. 24), a resonance condition that supports surface plasmon enhanced generation of radiation may be facilitated by one or more features that form a non-periodic structure together with an aperture in a planar conductive material such as a metal film.

In other embodiments, examples of a single feature in addition to an aperture to form such a resonance configuration include, but are not limited to, single non-annular topographic features such as another aperture, a depression in the metal film, and a protrusion (protuberance) that extends outwardly from the surface of the metal film. In one aspect, non-annular topographic features may have dimensions in cross-section on the order of the aperture. Like the single annular groove or raised ring discussed above in connection with FIG. 24, each of these single non-annular features causes a variation in a dielectric function along the surface of the metal film proximate to the aperture. Referring again to Eq. (3) above, and setting the index i equal to 1, according to the various embodiments discussed below a single non-annular feature proximate to an aperture positioned at a “resonant distance” αₒ from the aperture facilitates a resonance condition for surface plasmon enhance generation of radiation.

As noted above, in each of Eqs. (1) through (4), essentially normal incidence of the incident radiation to the irradiated surface of the metal film is assumed. It should be appreciated, however, that these equations may be appropriately modified to account for the effects of non-normal incidence on the maximum wavelength corresponding to a resonance condition. More generally, it should be appreciated that Eqs. (1) through (4) represent suitable working models for the observed resonance behavior of the corresponding resonance configurations, but that resonance behaviors may be more precisely modeled via a somewhat more complicated and detailed mathematical analysis of the underlying physics.

FIG. 26 illustrates varying the in-plane wave vector of photons incident on the illuminated surface of an SPEI apparatus by varying the angle of incidence of monochromatic light. For purposes of illustration, FIG. 26 depicts an SPEI apparatus 4200 having a resonance configuration comprising a linear array of apertures. However, as noted above in connection with FIG. 26 and Eq. (3), it should be appreciated that a similar resonance configuration may be realized by one aperture flanked by surface features that cause a variation in dielectric function proximate to the aperture. In the model of FIG. 26, any deviation from the angle normal to the resonance configuration of the SPEI apparatus (or multiple resonance configurations of an array of SPEI apparatus) increases in-plane momentum and therefore, the wave vector.

As described by Raether, Surface Plasmons on Smooth and Rough Surfaces and on Gratings. Springer Tracts on Modern Physics V 111. Springer Verlag 1988, and using the resonance configuration described by Eq. (3) above as an illustrative example, the relationship given in Eq. (5) specifies the wavelength λₘₐₓ at which the photons are resonant in an SPEI apparatus (based on a linear array of apertures or an aperture flanked by features) and an angle of incidence θ with respect to a normal to the surface of the resonance configuration(s):

$${λ}_{max} = \frac{{a}_{o}}{i} [ {( \frac{{ɛ}_{1} {ɛ}_{2}}{{ɛ}_{1} + {ɛ}_{2}} )}^{\frac{1}{2}} - sin θ ] . \qquad ( 5 )$$

As seen from the relation above, the wavelength at which the SPEI device is resonant varies as the angle of incidence θ (measured from the surface normal) varies. Thus, by increasing the incident angle θ from zero (for example, by moving the light source in the position indicated at 4201 to the position at 4203 as seen in FIG. 26), the wavelength at which an SPEI apparatus is resonant is split in λ_{max,1} and λ_{max,2} of which one is a higher energy than the normally incident case, λ_{max,0}. Note that the angle of incidence θ (measured from the surface normal) can be negative (e.g. the light may be directed downward and to the left from the right of normal as seen in FIG. 26), in which case the energy is decreased a result which is not apparent from the scalar equation above, but which can be shown from the vector form of the equation. This makes the array resonant at longer (shorter energy) wavelengths, so that a splitting of the resonant peaks exists.

It should be appreciated that Eqs. (1), (2), and (4) may be modified in a manner similar to that shown in Eq. (5) to account for non-normal irradiation of an SPEI apparatus or SPEI array. Varying the angle of incidence as illustrated in FIG. 26 may be used to better match a monochromatic light source to SPEI apparatus employed in microscopes, data storage devices and photolithography systems as discussed herein. In addition, by varying the angle of incidence over a range continuously or in discrete steps, and measuring the amount of illumination that passes through the sub-wavelength apertures of the SPEI apparatus as the angle changes, the performance of the SPEI apparatus may by characterized.

High Resolution, High Throughput Photolithography

According to other embodiments of the present disclosure, photolithography methods and apparatus may be implemented based on the exemplary SPEI apparatus configurations discussed above. In one embodiment, an optical system required to execute SPEI lithography is very simple; there are no reduction lenses or steering mirrors. All that is required is a somewhat mono-chromatic light source, such as a filtered broadband (e.g. Hg lamp) source or a laser, one or more (e.g., an array) of SPEI apparatus, a subnanometer translation stage (e.g. the nanopositioning systems available from Mad City Labs, Inc. of Madison, Wis.), a proximity sensor to maintain the SPEI apparatus at a proper photoresist distance, and a photoresist coated wafer.

Various techniques based on SPEI concepts may be used to improve the throughput of a lithographic process according to the present disclosure. First, a SPEI apparatus is used to achieve high light transmission to increase the speed at which the photoresist can be patterned. Other approaches increase the parallelism of the writing operation, as described below.

The first level of parallelism is achieved by the creation of a SPEI array that contains one emitting aperture for each IC on a wafer. The spacing between emitting apertures will be the same as the spacing between ICs on the wafer. By doing this, the same pattern can be written to all ICs at the same time. To achieve a level of stiffness that maintains the flatness of the device and therefore achieves a uniform device-to-photoresist spacing, a transmissive substrate may be prepared using the same techniques used to prepare semiconductor wafers and fabricate the SPEI device on the wafer. The SPEI device should match the index of refraction of the glass instead of air. The resulting wafer/SPEI device should be rigid enough to allow for a constant CD to be maintained; otherwise, the SPEI device would have to be farther from the photoresist and divergence of the emitted light will increase the minimum CD that can be achieved. Structural elements may be fabricated into the device to achieve the desired stiffness. The light source should provide uniform illumination over the wafer diameter.

The second level of parallelism is achieved by writing multiple features within an IC in parallel. This is achieved with two modifications to the system. First, “shutters” are added between the light source and the SPEI device. Second, an SPEI device is constructed that provides a palette of different shapes. The two basic shapes that would be included are a circular (or square) aperture and a line segment. Each of these shapes is preferably provided in different sizes (diameters for the circular apertures, and lengths and widths for the line segments), and the line segments preferably have different orientations (horizontal, vertical, +/−45°).

In one embodiment, the minimum shutter size is one consideration that may affect the density of emitting apertures. Shuttering the emission from portions of the device may be performed using a liquid crystal device to block the fight or locally affect the dielectric function of the good metal or by attaching wires to the individual resonant patterns in the device to alter the electron density and, hence, the resonance of the surface plasmons in the area local to the aperture in question, thereby controlling a pattern's emission.

By employing the concepts disclosed herein to create small illumination spot sizes, lithography employing surface plasmon enhanced illumination provides numerous advantages, including:
a) small spot size (2-50 nm) for enhanced resolution;
b) high throughput coupled with high resolution, making it particularly useful for semiconductor fabrication;
c) high light transmission;
d) no diffraction problems with masks as the critical dimensions and CDs are reduced
e) more flexible range the light wavelengths can be used, delivering high resolution light over a broad range of wavelengths (from deep ultraviolet well into the infrared range, supporting development of new photoresist chemistries for a variety of applications.
f) maskless production technology is compatible with rapid prototyping and low production volumes as well as high volume runs;
g) the cost and complexity of SPEI lithography are compatible with creation of a system that can be used for rapid prototyping of semiconductors, creation of high-resolution masks for e-beam and extreme UV, and other research uses of photolithography;
h) provides a general purpose tool to be used in non-semiconductor lithography applications in the fields of biology, drug discovery, and clinical diagnostics, including lithography applications such as biosensors, bio-patterning, and array detectors (DNA microarrays, protein and small molecule arrays), all of which that benefit greatly because SPEI can deliver small critical dimensions (CDs) without resorting to ultraviolet light that damages bio-molecules; and
i) further lithography applications such as MEMS, self-assembly, molecular electronics, and the study of physics phenomena at very small dimensions.

A “maskless” SPEI lithography system according to one embodiment of the present disclosure may employ a large-scale array of single beam SPEI apparatus fabricated on a solid substrate. These apparatus may be independently controlled, either by using a shutter or controlling individual light sources such as solid state laser diodes which irradiate the SPEI apparatus, to produce an array of switchable nanometric sources of light that are scanned over a wafer to directly write a pattern on the photoresist target.

The performance characteristics of SPEI devices that make a significant contribution in the improved lithography system include:
(1) Small illumination spot diameter: 52 nm illumination spot sizes can be obtained using 40 nm apertures, and the aperture diameter can be reduced to 5 nm-10 nm with currently available fabrication methods;
(2) The emitted light is propagating, not evanescent, thereby enabling the SPEI device-to-target distance to be on the order of 350 nm;
(3) The enhanced light transmission (emission/incident intensity of 3×) achieved with SPEI devices directly translates into higher throughput patterning;
(4) SPEI devices can be designed for a broad range of wavelengths, thus decoupling the feature size that can be patterned from the wavelength of the light. Devices have been designed and fabricated devices for wavelengths of 257 nm, 405 nm and 558 nm;
(5) SPEI technology is compatible with immersion lithography; and
(6) Maskless lithography using SPEI devices offers the promise of a simple stepper system.

Maskless SPEI-based photolithography systems can provide significantly improved performance in the following areas of the lithography industry:

Rapid prototyping: SPEI devices can be employed in devices used in R&D departments for rapid prototyping of integrated circuit designs and in device research to explore the performance of circuits at the nanoscale.

Mask fabrication using SPEI devices to directly write and pattern the masks: The direct write SPEI mask making system may use an array of independently controlled SPEI devices on a rigid substrate to write the mask pattern. Such a SPEI system is expected to maintain the throughput of current optical systems while extending the feature size well below what can currently be achieved with optical systems. This essentially combines the throughput of optical systems with the resolution of electron systems.

Stepper system for low throughput applications: A stepper system would build on the first two products described above and address ASICS (application specific integrated circuits) and military devices where the throughput demands are lower than commercial production. In addition to elimination of the mask costs, a stepper system is anticipated to be less expensive than current systems and capable of being deployed in a distributed manner.

Full production stepper system: The simplicity of SPEI based lithography yields a system that is much less expensive than current systems. As a result, a distributed approach may be used in production semiconductor fabrication by using a larger number of lower cost steppers. Maskless SPEI techniques may be integrated into existing stepper systems simply by modifying the optical system as discussed below.

For maskless photolithography, SPEI light funnel devices are preferably fabricated in an array and combined with a shuttering system or with independently controllable light sources to provide an array of independently controlled, bright nanometric sources of propagating light. This array of independently controlled light sources is scanned across a photoresist coated wafer while each nanometric light source is switched on and off to write a pattern in the photoresist with achievable feature sizes below 30 nm.

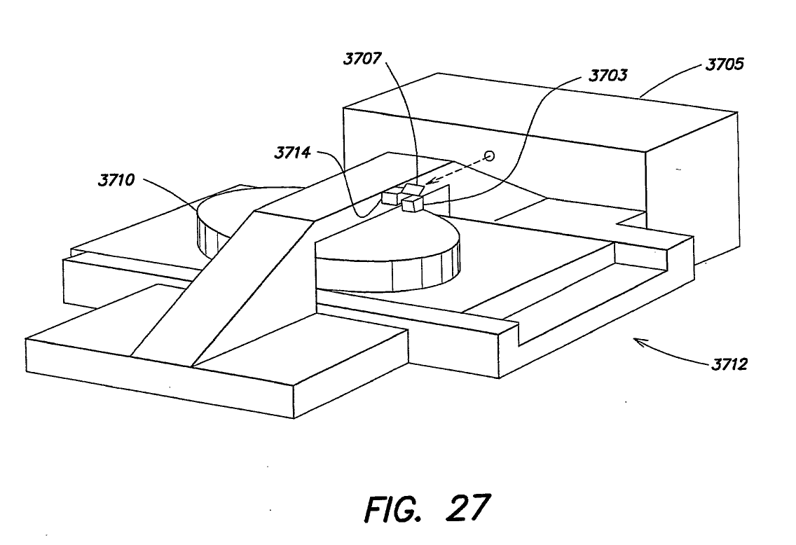

One embodiment of a maskless, direct write, SPEI photolithography system according to the present disclosure is shown in FIG. 27. In the arrangement shown, an array of SPEI apparatus in combination with a LCS light shutter is housed within a write engine seen at 3703. A large monochromatic laser 3705 directs a light beam via a folding mirror 3707 to the write engine 3703. A wafer coated with a photoresist is mounted on a chuck 3710 that is mounted for coarse movement with respect to the write engine on an X-Y stepper motor driven stage indicated generally at 3712. The write engine is mounted for fine movement on a piezoelectric nanometric positioner 3714.

Positioning devices such as the positioner 3714 capable of nanometer scale resolution typically employ piezo actuators to achieve controllable, nanometer-scale motion. Positioning is accomplished using the “inverse” piezoelectric effect, where a voltage applied to a certain crystalline structure will cause the crystal to change shape or expand. This expansion is proportional to the applied voltage and the configuration of the piezo element. Typical motion for an unlevered stacked piezoelectric element is on the order of 100 microns. To achieve greater travel, a stepper motor may be used to provide for coarse positioning, and piezo elements are used for fine positioning. Piezo stepper motor positioners are described, for example, in U.S. Pat. No. 6,150,750 issued to Burov et al. on Nov. 21, 2000 entitled “Piezoelectric linear step motor” and in U.S. Pat. No. 6,800,984 issued to Marth on Oct. 5, 2004 entitled “Piezo linear drive with a group of piezo actuator stacks as well as method for operating such a drive,” the disclosures of which are incorporated herein by reference. A variety of nanopositioning mechanisms suitable for implementing the disclosure are available from PI (Physik Instrumente) L. P., Auburn, Mass. 01501.

A schematic depiction of a single SPEI apparatus of the system of FIG. 27, irradiated by the switched light source, is shown in FIG. 28, while an array of such SPEI apparatus is illustrated in FIG. 29. A processor 3800 controls a switched light source 3801 that irradiates an SPEI apparatus indicated generally at 3802, wherein the SPEI apparatus includes a rigid transparent substrate 3803 and a metal film 3805. Light from the source 3801 is transmitted by surface plasmon resonance through the sub-wavelength aperture 3807 onto a photoresist 3822 on a wafer 3824 that is spaced from the SPEI apparatus. Dimples or other features in the illuminated surface of the metal film, one of which is indicated at 3813, provide enhanced light transmission through the aperture 3807. The transparent substrate 3803 has a different index of refraction than the air at the exit surface of the metal film 3805, decoupling the illuminated surface from the exit surface and suppressing the excitation of the surface plasmons on the exit surface. As a result, the illumination from the aperture 3807 indicated at 3830 is substantially collimated, exhibiting less than 1 degree of half angle divergence, so that the spot of illumination striking the photoresist is only slightly larger that the sub-wavelength aperture. The processor 3800 switches light source ON and OFF in accordance with the pixel image data 3840 as a nanometric positioner 3845 moves the photoresist/wafer relative to the SPEI aperture to write a virtual image onto the photoresist.

FIG. 29 illustrates a multi-channel device in which an array of SPEI apparatus is fabricated on a rigid substrate 3901. The SPEI array 3901 and the wafer indicated at 3903 are moved relative to one another in two dimensions by a stepper, nanometric positioner, or the like (not shown in FIG. 29) as the individual light sources irradiating corresponding SPEI apparatus are switched ON and OFF to directly write onto a photoresist on the upper surface of the wafer 3903. An arrangement of the type shown illustrated in FIG. 29 may be configured to write a single integrated circuit with each single SPEI aperture, in which case the positioner must be capable of a total travel in each direction at least equal to the size of the IC whose pattern is being written. Alternatively, each irradiated SPEI apparatus may write only a portion of a larger IC, and the positioner must have a total travel in each direction equal to the distances between the apertures in the SPEI array.

The switched light source 3801 seen in FIG. 28 may be a switched solid state laser, such as a VCSEL, that is formed as an integral part of the SPEI device structure. The VCSEL (Vertical Cavity Surface Emitting Laser) is a semiconductor microlaser diode that emits light in a cylindrical beam vertically from the surface of a fabricated wafer, and is widely used in fiber optic communications devices. VCSELs can be fabricated efficiently using standard microelectronic fabrication methods that allow integration of VCSELs on-board with other components, such as SPEI light funnels, without requiring pre-packaging. Arrays of VCSELs can be fabricated on a single substrate for integration with SPEI devices in a two-dimensional array configuration. VCSELs operate at low threshold currents that enable high-density arrays to be used. The surface normal emission of circular and low divergence output beams eliminate the need for corrective optics, and the light from a VCSEL can be directed onto the illuminated surface of an SPEI light funnel either directly or through focusing microlens. VCSEL arrays can be built and tested at the wafer level to provide low cost mass production. Moreover, VCSELs are capable of operating at very high switching speeds (above 1 Gigabit/sec) with low power consumption, permitting high speed direct write operations to be performed.

As seen in FIG. 30, each light source may be implemented as a separate vertical cavity surface emitting laser (VCSEL). Note that the VCSEL and SPEI structures shown in FIGS. 30 and 31 are not to scale and are, intended to portray the functional relationship between the parts and not their relative sizes. The VCSEL light source seen in FIG. 30 includes a substrate 4003 of a suitable semiconductor material, on which the other materials of the VCSEL can be grown, such as GaAs, Si, InP or the like, and is preferably of N-type conductivity. A transparent N-ohmic contact layer 4005 through which light is emitted is deposited onto lower surface of the substrate 4003. An SPEI light funnel device consisting of the transparent dielectric substrate 4007 which supports a metal film 4010 is attached to the VCSEL substrate 4003. The upper illuminated surface of the metal film is shaped to define an aperture 4007 which passes through the metal film 4010 is surrounded by a concentric bullseye pattern consisting of a pair of annular indentations 4008 in the surface of the film 4010 (which do not go all the way through the metal film). A first mirror stack indicated generally at 4012 is a distributed Bragg reflector and is formed of alternate layers of semiconductor materials having different indices of refraction. An insulator 4015 above the mirror stack 4012 extends up and around an active layer 4021 and second mirror stack 4023. The active layer 4021 is sandwiched between cladding that separates it from the mirror stacks 4012 and 4023. The second mirror stack 4023 is also a distributed Bragg reflector and, like the first mirror stack 4012, comprises alternating layers of materials having different indices of refraction. A heat sink 4040 consisting of a body of thermally conductive material is mounted on the contact layer 4042 by a suitable thermal bonding material.

Vertical cavity surface emitting laser (VCSEL) devices which may used as the light source for controllable illuminating arm SPEI “light funnel” may be readily fabricated in arrays as shown in FIG. 30. Suitable VCSEL arrays are described in “Micro-Optical 2-D Array Devices For Optical Disk Head Of Ultra-High Data Rate And Density Using VCSEL” by Kenya Goto, 8th Microoptics Conference (MOC '01), Osaka, Japan, Oct. 24-26, 2001; in the paper “Highly Uniform Vertical-Cavity Surface-Emitting Lasers Integrated with Microlens Arrays,” by S. Eitel et al., IEEE Photonics Technology Letters, Vol. 12, No. 5, May 2000; and in U.S. Patent Application Publication 2005-0025211 by Zhang et al. entitled “VCSEL and VCSEL array having integrated microlenses for use in a semiconductor laser pumped solid state laser system.” The VCSEL arrays described in the foregoing references both use microlens structures for focusing the light emitted by the laser and, in both cases, these microlens structures may be retained to focus light on the illuminated metal film surface of the SPEI device, or the microlens structure may be eliminated and replaced by the SPEI light funnel as shown in FIG. 30.

Edge emitters, called HCSELs (horizontal cavity SELs) may be used to particular advantage in combination with SPEI structures in photolithography applications. These devices have higher power, are more efficient, substantially less expensive (because of better yields) and, most importantly, emit light all the way down to the violet end of the spectrum, avoiding the need for VNIR or red photoresists and the associated changes to the cleanroom. One and two dimensional HCSEL diode arrays are available from BinOptics Corporation, 9 Brown Road, Ithaca, N.Y. 14850.

In a further alternative configuration illustrated in FIG. 31, the transparent dielectric substrate 4107 attached to a perforated metal film 4010 may be shaped to form a lenslet over the aperture and surface pattern that forms each light funnel. The lenslet gathers light from the larger VCSEL and focuses it on the corrugated features of the illuminated surface of the SPEI light funnel.

When SPEI devices are used in a maskless lithography system, the feature size that can be achieved is not constrained by the wavelength of the light or the numerical aperture of the illumination lens. Instead the feature size is governed by the size of the central emitting aperture and the distance between the SPEI device and the target. The surface features and dielectric constants of the SPEI are designed for resonance and enhanced light transmission through the aperture at the specific wavelength of the light source and to suppress the excitation of surface plasmons on the exit surface to achieve a collimated directed onto the photoresist target. The selection of the metals from which the device is constructed and the lattice constant (spacing between the corrugations) are the two primary design variables, while some additional modulation of the wavelength is available by controlling and adjusting the angle of incidence as discussed in connection with FIG. 23.

The ability to design the SPEI device for use at a desired wavelength provides more flexibility in the selection and/or development of new photoresists with SPEI than with other lithography approaches. Because SPEI can achieve its critical dimension limits with any wavelength, the only constraint imposed on the photoresist is that its inherent resolution (grain size and catalytic activity) be compatible with the desired critical dimension.

Large-scale SPEI photolithography is compatible with existing stepper systems modified to mount the SPEI substrate and using a modified optical system. The key requirements for the optical system are that an array of individually controlled collimated light beans of the correct wavelength be delivered to the illumination surface of the SPEI array.

The individual light beams may be controlled using digital micromirrors such as the digital mirror device (DMD) spatial light modulator described in U.S. Pat. No. 5,515,076 issued to Thompson et al. (Texas Instruments) on May 7, 1996, the disclosure of which is incorporated herein by reference.

Other forms of spatial light modulators, such as LCD panels, may be employed to control the magnitude of light applied to, or emitted by, the individual SPEI apparatus. An introductory description of the construction, features, and use of liquid crystal displays and the manner in which they may be driven by integrated circuits and processors is presented in “LCD Fundamentals using PIC16C92X Microcontrollers,” Datasheet No. DS00658A, 1997, Microchip Technology, Inc., and in U.S. Pat. No. 5,861,861 issued to Nolan, et al. on Jan. 19, 1999 entitled “Microcontroller chip with integrated LCD control module and switched capacitor driver circuit,” the disclosure of which is incorporated herein by reference.

The grating light valve (GLV) is still another form of spatial light modulator that may be employed to control the amount of light flowing through each SPEI light funnel in a direct write lithography system. GLV devices are described in “Overview and applications of Grating Light Valve™ based optical write engines for high-speed digital imaging,” by Jahja T. Trisnadi et al (Silicon Light Machines), Photonics West 2004—Micromachining and Microfabrication Symposium, Jan. 26, 2004, San Jose, Calif., USA and in U.S. Pat. No. 6,466,354 issued to Gudeman (Silicon Light Machines) on Oct. 15, 2002 entitled “Method and apparatus for interferometric modulation of light, the disclosure of which is incorporated herein by reference.

Four exemplary systems which represent photolithography applications of the concepts disclosed herein are described and compared below. It should be appreciated that these examples are provided primarily for illustrative purposes, and that the present disclosure is not limited to these particular examples.

SYSTEM 1: The first system utilizes a configuration of the kind illustrated in FIG. 31 in which the SPEI light funnels are fabricated as part of a lenslet array. An array of 13,800 individually controlled VCSEL lasers operating at VNIR or red wavelengths are packed together with a center-to-center spacing of 115 μm. The SPEI array likewise consist of 13,800 individual patterns each consisting of an aperture with adjacent surface irregularities, such as an aperture positioned in a line of dimples which resonate polarized light from an adjacent solid state laser. The patterns may be fabricated by FIB milling or electron beam photolithography directly on the lenslet array which acts as a substrate and provides the appropriate dielectric constant for achieving collimated light output directed to the photoreceptor target. The photoreceptor/wafer is moved relative to the controlled SPEI light source array using a scanner/stepper stage that moves with a 200 cm/s average velocity on fine movement and a 0.005 s settling time on coarse movement

Exemplary System 1 employs existing VCSEL light sources and associated electronic drive circuitry to achieve very high switching speeds during writing (the VCSELs support switching rates of 1 GHz. and higher). The alignment of the VCSEL array, the lenslet array, and the SPEI array is not difficult. The system employs minimal optics components and is very compact. SYSTEM 1 can be made at the lowest cost of the four systems example systems and can provide a throughput of 80 wafers/hour. It should be appreciated that System 1 requires a red or VNIR photoresist to match the wavelength of the VCSEL light sources, with a potential need to alter the lighting at the fabrication facility. System 1 also requires a scanner/stepper capable of high velocity and high precision.

SYSTEM 2. The second example system is configured as illustrated in FIG. 30 with the SPEI light funnel array being fabricated directly on the VCSEL array. The VCSEL array consists of 444,000 individually controlled lasers operating at VNIR or red wavelengths with a 30 μm center to-center packing density. The SPEI array of 444,000 individual bull's eye type patterns is fabricated using FIB milling or electron beam photolithography and fabricated directly onto VCSEL array. The VCSEL/SPEI array structure is translated relative to the photoresist/wafer using a PI Picocube and air bearing stage with interferometric feedback that provides 200 cm/sec average velocity on fine movement, a total travel of 5 μm, and 0.005 s settling time on coarse movement the PI Picocube and other nanopositioning mechanisms are available from PI (Physik Instrumente) L. P., Auburn, Mass. 01501.

Exemplary System 2 employs existing VCSEL light sources and associated electronic drive circuitry to achieve very high switching speeds during writing (the VCSELs support switching rates of 1 GHz. and higher). The system employs minimal optics components and is very compact. It should be appreciated that System 2 requires a red or VNIR photoresist to match the wavelength of the VCSEL light sources, with a potential need to alter the lighting at the fabrication facility. It does not make the most efficient use of the VCSELs, and the high packing density of the SPEI array may create heating problems that limit its use to low power densities, potentially decreasing its useful speed. Like System 1, in requires the use of a red/VNIR photoresist and may require a change in the lighting of fabrication facility.

SYSTEM 3. The third example system is configured as illustrated in FIG. 31 with the SPEI array of 5 million light funnels fabricated as part of a lenslet array. System 3 employs a single green (532 nm), 18 watt, continuous-wave laser operating with a 2D grating light valve providing 5 megapixel resolution. The light illuminating each SPEI light funnel is controlled by a 2D, 5 megapixel Grating Light Valve that exhibits an optical efficiency of 0.38. The lenslet and SPEI device array of 5 million lenses and SPEI light funnels is constructed with a 2.5 um center to center spacing. System 3 uses a PI Picocube nanopositioning system and air bearing stage with interferometric feedback providing 200 cm/s average velocity on fine movement, 5 μm total travel, and 0.005 s settling time on coarse movement.

In System 3, the structure of the lenslet/SPEI array allows off of the shelf movement components. The use of the green CW laser eliminates the need to develop or use a special photoresist. The single laser source provides more efficient illumination of SPEI array (low heating of SPEI array) with good thermal performance due to good irradiance efficiency.

SYSTEM 4. The fourth example system employs eleven blue (473 nm), 1 watt, CW lasers in combination with a 2D Grating Light Valve (5 megapixel) providing 0.38 optical efficiency. The SPEI array of 5 million bullseye patterns is fabricated directly on the lenslet array as in FIG. 31 and uses a 2.5 um center to center spacing. System 4 also uses a PI Picocube nanopositioning system and air bearing stage with interferometric feedback providing 200 cm/s average velocity on fine movement, 5 μm total travel, and 0.005 s settling time on coarse movement.

In exemplary System 4, the structure of the lenslet/SPEI array allows off of the shelf movement components. The use of the blue CW laser eliminates the need to develop or use a special photoresist. The eleven lasers provide efficient illumination of SPEI array (low heating of SPEI array) with good thermal performance due to good irradiance efficiency.

Many of the components, features and considerations discussed above with respect to SPEI photolithography are also applicable to other applications which benefit from the advantages provided by SPEI devices. SPEI optical data storage systems discussed above in connection with FIGS. 21, 21A, 21B and 21C may use the SPEI arrays and switched light sources discussed above for use in direct write photolithography. Similarly, the SPEI array and nanometric movement mechanisms may be employed in surface plasmon enhanced microscopy as described above in connection with FIG. 22.

SPEI photolithography may be employed as a manufacturing method for a binding biosensor or nucleic acid microarray in which the density of nucleic acid probes substantially exceeds the density that can be achieved using traditional photolithography methods that are limited by the Rayleigh criterion. SPEI lithography can also be used for any type of array sensor where photochemistry is used to prepare the surface for immobilization of a ligand or in situ synthesis of the ligands. For example, in very large scale immobilized polymer synthesis systems, a substrate having positionally defined oligonucleotide probes is synthesized. See, for example, Pirrung et al. U.S. Pat. Nos. 6,416,952, 5,143,854; and 5,489,678. In these prior arrangements, conventional projection photolithographic using masks with UV illumination is used in combination with photosensitive synthetic subunits for the stepwise synthesis of polymers according to a positionally defined matrix pattern. Each oligonucleotide probe is thereby synthesized at known and defined positional locations on the substrate. However, the density of the array is constrained by the conventional photolithography-methods whose resolution is limited by the Rayliegh criterion. By using SPEI) this synthesis process may be performed using a direct-write method, eliminating the need to create a mask, and providing significantly improved probe density. Direct write is the equivalent of using a paint brush to paint a picture whereas projection lithography with masks is akin to silk screening the picture. Silk screening, when it is compatible with the resolution required is faster. However, the masks in photolithography are expensive and they wear out. This will increasingly be a serious problem for the semiconductor industry as the feature sizes decrease the cost of the masks increase and their lifetime decrease.

It will be apparent to one skilled in the art that the use of the disclosure for photolithography extends to all photochemical applications where a pattern is created, as photolithography is a specific field of photochemistry. This would include the preparation of surfaces for subsequent operations and/or chemical reactions, or the creation of micro- or nano-reaction vessels in which the chemical reaction is caused or promoted or inhibited by the addition of light.

It is to be understood that the specific embodiments and applications of the concepts disclosed herein are merely illustrative. Numerous modifications may be made to the methods and apparatus described without departing from the true spirit and scope of the disclosure.

1. An apparatus, comprising:
a plurality of surface emitting lasers (SELs); and
a plurality of surface plasmon enhanced illumination (SPEI) apparatus disposed with respect to the plurality of SELs such that:
a first SEL of the plurality of SELs, configured to generate first radiation, irradiates at least a first SPEI apparatus of the plurality of SPEI apparatus when the first radiation is generated; and
a second SEL of the plurality of SELs, configured to generate second radiation, irradiates at least a second SPEI apparatus of the plurality of SPEI apparatus when the second radiation is generated.

2. The apparatus of claim 1, wherein the plurality of SPEI apparatus are arranged as an array of SPEI apparatus fabricated on a rigid substrate.

3. The apparatus of claim 2, wherein the array of SPEI apparatus comprises a two-dimensional array.

4. The apparatus of claim 1, wherein each of the first and second SPEI apparatus comprises:
a metal film having a first surface and a second surface; and
at least one resonance configuration formed in the metal film, the at least one resonance configuration comprising:
an aperture extending between the first surface and the second surface of the metal film; and
at least one feature that causes a variation in a dielectric function along the first surface of the metal film proximate to the aperture, wherein the aperture and the at least one feature are configured so as to cooperatively facilitate a resonance condition for surface plasmon enhanced radiation generated by the SPEI apparatus, based on incident radiation, when present, that irradiates the first surface of the metal film.

5. The apparatus of claim 4, wherein the at least one feature includes a plurality of features that forms a periodic structure together with the aperture.

6. The apparatus of claim 4, wherein the at least one feature includes at least one dimple.

7. The apparatus of claim 4, wherein the at least one feature includes at least one annular groove or at least one raised ring surrounding the aperture.

8. The apparatus of claim 4, wherein the at least one feature includes a pair of annular indentations forming a concentric bulls-eye pattern surrounding the aperture.

9. The apparatus of claim 4, wherein the at least one feature forms a non-periodic structure together with the aperture.

10. The apparatus of claim 4, wherein the at least one feature comprises a single feature.

11. The apparatus of claim 10, wherein the single feature comprises a single annular groove or a single raised ring around the aperture.

12. The apparatus of an of the foregoing cla claim 1, wherein the first and second SELs respectively comprise first and second vertical cavity surface emitting lasers (VCSELs).

13. The apparatus of claim 12, wherein each VCSEL of the first and second VCSELs comprises:
a semiconductor substrate; and
a transparent contact layer through which radiation is emitted from the VCSEL.

14. The apparatus of claim 13, wherein:
the first and second VCSELs are positioned to respectively irradiate only the first and second SPEI apparatus of the plurality of SPEI apparatus; and
each of the first and second SPEI apparatus includes a transparent substrate coupled to the transparent contact layer of a corresponding VCSEL, such that the plurality of switched light sources and the plurality of SPEI apparatus form an integrated structure.

15. The apparatus of 13, wherein:
the first and second VCSELs are positioned to respectively irradiate only the first and second SPEI apparatus of the plurality of SPEI apparatus; and
each of the first and second SPEI apparatus includes a transparent substrate shaped to form a lenslet to focus radiation emitted by a corresponding VCSEL.

16. The apparatus of claim 1, wherein the first and second SELs respectively comprise horizontal cavity surface emitting lasers (HCSELs).

17. The apparatus of claim 1, further comprising at least one processor coupled to at least the first and second SELs and configured to independently control at least the first radiation and the second radiation.

18. The apparatus of claim 17, further comprising:
a stage for supporting a wafer; and
a positioner responsive to at least one control signal generated by the at least one processor and configured to vary a relative position between the stage and surface plasmon enhanced radiation generated by the plurality of SPEI apparatus.

19. The apparatus of claim 18, wherein the at least one processor is configured to independently control the first and second radiation, and vary the relative position between the stage and the surface plasmon enhanced radiation, based at least in part on pixel image data.

20. A photolithography method, comprising:
generating first radiation from a first surface emitting laser (SEL);
irradiating at least a first surface plasmon enhanced illumination (SPEI) apparatus with the first radiation so as to generate first surface plasmon enhanced radiation;
generating second radiation from a second surface emitting laser (SEL);
irradiating at least a second surface plasmon enhanced illumination (SPEI) apparatus with the second radiation so as to generate second surface plasmon enhanced radiation; and
exposing a photoresist to the first surface plasmon enhanced radiation and the second surface plasmon enhanced radiation.

21. The method of claim 20, further comprising independently controlling the first and second radiation.

22. The method of claim 21, further comprising:
positioning the photoresist relative to the first surface plasmon enhanced radiation and the second surface plasmon enhanced radiation based at least in part on pixel image data.

23. The method of claim 20, wherein the first and second SELs respectively comprise vertical cavity surface emitting lasers (VCSELs).

24. The method of claim 20, wherein the first and second SELs respectively comprise horizontal cavity surface emitting lasers (HCSELs).
